(19) United States
(12) Patent Application Publication
(10) Pub. No.: US 2002/0033454 A1
(43) Pub. Date: Mar. 21, 2002

(54) Optical imaging system with direct image construction

(76) Inventors: Xuefeng Cheng, Milpitas, CA (US); Xiaorong Xu, Menlo Park, CA (US); Shuoming Zhou, Cupertino, CA (US); Lai Wang, Cupertino, CA (US); Ming Wang, San Jose, CA (US); Feng Li, San Jose, CA (US); Guobao Hu, San Jose, CA (US)

Correspondence Address: Pennie & Edmonds, LLP, 3300 Hillview Avenue, Palo Alto, CA, 94304, US

(21) Appl. No.: 09/778,617

(22) Filed: Feb. 6, 2001

Related U.S. Application Data
(60) Provisional application No. 60/223,074, filed on Aug. 4, 2000.

(51) Int. Cl. G01J 5/02; A61B 5/00

(52) U.S. Cl. 250/339.12; 600/322

(57) ABSTRACT

The invention generally relates to optical imaging systems and methods for providing images of two-dimensional or three-dimensional spatial or temporal distribution of properties of chromophores in a physiological medium. More particularly, the following description provides preferred embodiments of optical imaging systems utilizing efficient, real-time image construction algorithms. A typical optical imaging system includes at least one wave source, at least one wave detector, a movable member, an actuator member, and an imaging member. The wave source emits electromagnetic waves into a target area of the medium, and the wave detector detects electromagnetic waves and generates output signal in response thereto. The movable member includes the wave source and/or detector, and the actuator member moves the movable member along with the wave source and detector over different regions of the target area while the wave detector generates the output signal therefrom. The imaging member generates a set of voxels in the target area and calculates voxel values each of which represents a spatial or temporal average of the property of the chromophore in each voxel. The imaging member generates a set of cross-voxels from the intersecting voxels, and calculates cross-voxel values of the cross-voxels directly from the voxel values of the intersecting voxels. The imaging member then constructs the images of the chromophore properties in the target area. Accordingly, without needing to resort to the time-consuming conventional image reconstruction methods, the optical imaging system of the present invention can construct such images on a substantially real time basis.

### CROSS-REFERENCE TO RELATED APPLICATIONS

This application claims the benefit of the filing date of U.S. Provisional Patent Application bearing Ser. No. 60/223,074, entitled “A Self-Calibrated Optical Scanner for Diffuse Optical Imaging” filed on Aug. 4, 2000.

### FIELD OF THE INVENTION

The present invention generally relates to an optical imaging system and methods thereof for providing images representing spatial and/or temporal distribution of chromophores or their properties in a physiological medium. More particularly, the present invention relates to a non-invasive optical imaging system equipped with real-time image construction algorithms and methods thereof. The present invention is applicable to optical imaging systems whose operation is based on wave equations such as the Beer-Lambert equation, modified Beer-Lambert equation, photon diffusion equation, and their variations.

### BACKGROUND OF THE INVENTION

Near-infrared spectroscopy has been used to measure various physiological properties in animal and human subjects. The basic principle underlying the near-infrared spectroscopy is that a physiological medium such as tissues and cells includes a variety of light-absorbing and light-scattering chromophores which can interact with electromagnetic waves transmitted thereto and traveling therethrough. For example, human tissues include numerous chromophores among which deoxygenated and oxygenated hemoglobins are the most dominant chromophores in the spectrum range of 700 nm to 900 nm. Therefore, the near-infrared spectroscopy has been applied to measure oxygen levels in the physiological medium in terms of tissue hemoglobin oxygen saturation (or simply “oxygen saturation” hereinafter). Technical background for the near-infrared spectroscopy and diffuse optical imaging has been discussed in, e.g., Neuman, M. R., “Pulse Oximetry: Physical Principles, Technical Realization and Present Limitations,” Adv. Exp. Med. Biol., vol. 220, p.135-144, 1987 and Severinghaus, J. W., “History and Recent Developments in Pulse Oximetry,” Scan. J Clin. and Lab. Investigations, vol. 53, p.105-111, 1993.

Various techniques have been developed for the near-infrared spectroscopy, including time-resolved spectroscopy (TRS), phase modulation spectroscopy (PMS), and continuous wave spectroscopy (CWS). In a homogeneous, semi-infinite model, the TRS and PMS are generally used to solve the photon diffusion equation, to obtain the spectra of absorption coefficients and reduced scattering coefficients of the physiological medium, and to estimate concentrations of the oxygenated and deoxygenated hemoglobins and oxygen saturation. To the contrary, the CWS has generally been used to solve the modified Beer-Lambert equation and to calculate relative values of or changes in the concentrations of the oxygenated and deoxygenated hemoglobins.

Despite their capability of providing hemoglobin concentrations as well as the oxygen saturation, the major disadvantage of the TRS and PMS is that the equipment has to be bulky and, therefore, expensive. The CWS may be manufactured at a lower cost but is generally limited in its utility, for it can estimate only the changes in the hemoglobin concentrations but not the absolute values thereof. Accordingly, the CWS cannot provide the oxygen saturation. The prior art technology also requires a priori calibration of optical probes before their clinical application by, e.g., measuring a baseline in a reference medium or in a homogeneous region of the medium of a test subject. Furthermore, all prior art devices have to adopt complicated image reconstruction algorithms to generate images of two-dimensional or three-dimensional distribution of the chromophore properties.

Accordingly, there exist needs for compact and relatively cheap optical imaging systems incorporating more efficient image construction algorithms and capable of providing two-dimensional or three-dimensional images of distribution of chromophores on a substantially real time basis.

### SUMMARY OF THE INVENTION

The present invention generally relates to optical imaging systems, optical probes, and methods thereof for providing two- or three-dimensional images of spatial or temporal distribution of properties of chromophores in various physiological media. More particularly, the present invention relates to novel optical imaging systems equipped with mobile scanning units or movable sensor assemblies, real time baseline estimation and self-calibration algorithms, and real-time image construction algorithms.

In one aspect of the present invention, an optical imaging system generates images of a target area of a physiological medium, where such images generally represent distribution of at least one property of at least one chromophore in the medium. A typical optical imaging system includes at least one wave source for irradiating electromagnetic waves into the medium and at least one wave detector for detecting electromagnetic waves and generating output signal in response thereto. The optical imaging system also includes at least one movable member having at least one of the wave source and detector therein, and an actuator member operationally coupling with the movable member and generating one or more movements of the movable member with respect to the target area along one or more curvilinear paths.

The optical imaging system of the invention offers numerous benefits over the prior art technologies. Contrary to the conventional optical imaging equipment which allows a single measurement at each measurement location, the optical imaging system of the present invention provides a scanning unit or sensor assembly (generally referred to as “scanning unit” hereinafter) which is positioned at one region of a much larger target area and moves through other regions of the target area without moving other components of the optical imaging system toward other measurement regions of the target area. Accordingly, the foregoing optical imaging system can scan the target area which may be many times larger than a single scanning area of the scanning unit. The optical imaging system of the present invention requires fewer parts (i.e., fewer wave sources and/or detectors) than its conventional counterparts. Thus, the foregoing optical imaging system can be constructed as a light and compact system which can be portably worn by the test subject. In addition, such optical imaging system may minimize signal noises and image glitches attributed to idiosyncratic component variances inherent in each wave source and detector. As a result, the foregoing optical imaging system can generate output signals having improved signal-to-noise ratios and provide images with higher resolution therefrom . The optical imaging system of the present invention ensures that appropriate optical couplings are maintained between the medium and movable sensors such as the wave sources and detectors during the movement of the movable member. Accordingly, a single baseline may be obtained from the medium and used to normalize the input and output signals obtained in different regions of the target area and in different target areas of the medium. The optical imaging system of the present invention further provides real-time images of the distribution of chromophore property by employing much simpler and more efficient image construction algorithms.

Embodiments of this aspect of the present invention may include one or more of the following features.

The actuator member moves the movable member by generating movements such as curvilinear translations, rotations, revolutions, and/or reciprocations at a constant or variable speed. Such movements may be impulses, steps, pulses, pulse trains or sinusoids, each of which may be repeated periodically or at pre-determined intervals.

The wave sources and detectors provide one or more scanning units each of which includes a longitudinal axis extending through the wave source and detector and defines a scanning area therearound. Thus, the scanning units can move with the movable member while defining multiple scanning areas along the curvilinear path of the movable member. The wave sources and detectors are preferably arranged so that the scanning areas of their scanning units occupy only a portion of the target area. The wave sources and/or detectors may also be arranged to align their scanning units substantially orthogonal to or parallel with at least a portion of the curvilinear path of the movable member. In addition, the wave sources and/or detectors may be aligned so that their scanning units form pre-determined angles with the curvilinear path of the movable member.

The movable member may have at least two wave detectors at least two of which are disposed substantially linearly along the longitudinal axis of the scanning unit. The movable member may also include at least two wave sources at least two of which are disposed substantially linearly along the same axis. In addition, at least two wave detectors (or sources) may be interposed between at least two wave sources (or detectors) at identical distances. In the alternative, the movable member may include at least two wave sources and at least two wave detectors, where a first wave source (or detector) is disposed on one side across a line connecting the wave detectors (or sources) and where a second wave source (or detector) is disposed on the other side across the same line. The first and second wave sources (or detectors) may be arranged substantially symmetrically with respect to a line of symmetry or symmetrically with respect to a point of symmetry.

The actuator member may generate at least two movements of the movable member along one or more curvilinear paths either sequentially or simultaneously. The curvilinear paths may be arranged so that at least a portion of one curvilinear path may be substantially parallel with or orthogonal to at least a portion of the other curvilinear path, such as the orthogonal axes of the Cartesian, cylindrical, and spherical coordinate systems. The actuator member may generate a first movement of the movable member from a first region of the target area toward a second region thereof and then a second movement from the second region toward the first region of the target area. In case the target area should be substantially polygonal, the actuator member may generate multiple movements of the movable member sequentially, e.g., by generating a first movement of the movable member from a first side toward a second side of the target area, a second movement thereof from the second side toward a third side of the target area, and a third movement thereof from the third side toward the first or another side of the target area. The actuator member may also generate a first and second movements of the movable member simultaneously along a first and second curvilinear paths, respectively, where at least a portion of the first curvilinear path may be arranged to be substantially parallel with or orthogonal to at least a portion of the second curvilinear path. For example, the first movement may be a linear translation, while the second movement may be a linear reciprocation.

In another aspect of the present invention, an optical imaging system may include at least one of the foregoing wave sources, at least one of the foregoing wave detectors, a movable member, an actuator member, and an imaging member. The actuator member operationally couples with the movable member and generates movements of the movable member with respect to the target area of the medium along at least one curvilinear path. The imaging member receives and samples the output signals generated by the wave detectors, determines the chromophore property by solving wave equations applied to the wave sources and detectors, and generates the images representing the distribution of the chromophore property. Examples of such wave equations may include, but not limited to, the Beer-Lambert equation, modified Beer-Lambert equation, photon diffusion equation, and their variations.

Embodiments of this aspect of the present invention may include one or more of the following features.

The imaging member generally defines a set of a plurality of voxels in the target area. Each voxel includes a voxel axis and has a characteristic dimension which may be one of a voxel height, length, and width. Such voxels are sequentially arranged along a voxel direction which is generally parallel with the curvilinear path of the movable member. Such voxels may also align their axes substantially parallel with the longitudinal axis of the scanning unit, and may have heights substantially similar to that of the scanning unit.

The imaging member may include a data acquisition unit, signal analyzer or signal processor to receive and sample the output signals from the wave detectors at a pre-selected time interval or at pre-determined positions over the target area. The characteristic dimension of the voxels may be at least partially proportional to the speed of the movement of the movable member and/or inversely proportional to a sampling rate or data acquisition rate of the output signals by the data acquisition unit.

The imaging member may also generate a sequence of voxel values which are arranged in the same order as the voxels along the voxel direction, where each of such voxel values generally represents an average of the chromophore property in an area or a volume of each voxel. Each voxel value is typically obtained by solving the wave equations applied to the wave sources and detectors which define the voxel for such voxel value. In particular, when the actuator member generates at least two movements of the movable member along one or more curvilinear paths, the imaging member defines, during each of such movements, one set of the foregoing voxels and one corresponding sequence of the foregoing voxel values. When the imaging member defines multiple sets of voxels which extend in more than one voxel direction and which, therefore, intersect each other, the imaging member also defines cross-voxels, i.e., the overlapping or intersecting portions of at least two intersecting voxels each of which belongs to a different set of such voxels and each of which extends along a different voxel axis. The imaging member then generates a sequence of cross-voxel values “directly” from the voxel values of the intersecting voxels by obtaining, e.g., arithmetic, geometric, weighted- or ensemble-averages or sums of the voxel values of the intersecting voxels.

The imaging member may be arranged to identify at least one portion of the output signal having a substantially flat profile and similar magnitudes which may vary within a pre-selected range of deviation or which may be greater or less than a pre-selected cut-off magnitude. The imaging member calculates a baseline of the output signal by obtaining an average magnitude throughout such flat portion of the output signal. The imaging member normalizes the output signal by, e.g., providing an optical density signal defined as a ratio of a difference between the output signal and the baseline to the baseline.

In yet another aspect, an optical imaging system includes at least one sensor assembly with at least one of the foregoing wave sources and at least one of the foregoing wave detectors, a body arranged to support at least a portion of the sensor assembly, and an actuator member operationally coupling with at least one of the sensor assembly and body and arranged to generate at least one movement of at least one of the sensor assembly and body with respect to the target area of the medium along at least one curvilinear path.

Embodiments of this aspect of the present invention may include one or more of the following features.

The sensor assembly fixedly couples with the body so that the actuator member moves both of the body and sensor assembly in unison with respect to the target area of the medium. In the alternative, the sensor assembly may movably couple with the body so that the actuator member generates a first movement of the sensor assembly with respect to the body and target area as well as a second movement of the body with respect to the target area. The actuator member may generate at least a portion of the first movement simultaneously with at least a portion of the second movement. Alternatively, the actuator member may generate the first and second movements sequentially. The body may include a moving unit capable of moving both the sensor assembly and body to different target areas of the medium. In addition, the sensor assembly and/or body may be constructed as a hand-held optical probe or as a portable probe.

In a further aspect of the invention, an optical imaging system includes at least one portable probe and a console. The portable probe generally includes at least one movable member and an actuator member. The movable member includes at least one of the foregoing wave sources and at least one of the foregoing wave detectors. The actuator member operationally couples with the movable member and generates movements of the movable member along at least one curvilinear path. The console includes an imaging member arranged to receive and sample the output signal, to determine the property of the chromophore by solving wave equations applied to the wave source and detector, and to generate images of the two- or three-dimensional distribution of the chromophore property.

The foregoing aspect of the present invention offers benefits over the prior art counterparts. First, bulky or heavy components can be disposed in the console, while only essential elements are included in the portable probe. Therefore, the portable probe can maintain a compact size and light weight. Secondly, because such portable probe needs fewer components, idiosyncratic errors due to component variances may also be minimized. Thirdly, such probe may be constructed as a semi-portable article which can be worn by a patient for constantly or periodically monitoring the chromophore property thereof and for constructing images therefrom.

Embodiments of this aspect of the present invention may include one or more of the following features.

The optical imaging system includes at least one connector member capable of providing, e.g., electrical or optical communication of signals, electrical or mechanical power transmission, and/or data transmission between the portable probe and the console. The portable probe may include a rechargeable power supply unit and be made as a separate article which may be detached or detachable from the console. The portable probe may be arranged to transmit the output signal to the console telemetrically or may include a memory member capable of storing data such as the output signals generated by the wave detector.

In yet another aspect, an optical imaging system includes at least one of the foregoing wave source, at least one of the foregoing wave detectors, at least one optical probe, a console, an actuator, and a connector member. The optical probe includes at least one movable member, while the console operationally couples with the optical probe and includes an imaging member which receives and samples the output signal, determines the chromophore property, and generates images representing two- or three-dimensional spatial distribution of such property. The actuator member operationally couples with the movable member and generates at least one movement of the movable member along at least one curvilinear path. The connector member provides electrical or optical communication of various signals, electrical or mechanical power transmission or signal or data transmission between the optical probe, console, and/or actuator member.

Embodiments of this aspect of the present invention may include one or more of the following features.

In general, the movable member is arranged to include the wave source and detector therein. However, all of the wave sources and detectors may be included in the console as well. In such an embodiment, the connector member is provided with at least one optical pathway which optically couples the optical probe with the wave source and/or detector in the console, thereby transmitting electromagnetic waves therebetween. A typical example of such optical pathway is a fiber optics product such as an optical fiber.

Similarly, at least one actuator member may be disposed at the optical probe and/or console. For example, the actuator member may be incorporated into the console, and the connector member is provided with a mechanical power transmission pathway arranged to transmit the mechanical power from the actuator member toward the optical probe therethrough.

In yet another aspect of the invention, an optical imaging system includes at least two wave sources arranged to irradiate electromagnetic waves into the medium and at least two wave detectors arranged to generate output signals responsive to electromagnetic waves detected thereby. At least two of such wave sources and at least two of such wave detectors may be disposed substantially linearly along a straight line.

Embodiments of this aspect of the present invention may include one or more of the following features.

All of the wave sources and detectors may be disposed substantially linearly along the straight line. An actuator member may generate one or more movements of at least one of the wave sources and detectors or, in the alternative, all of the wave sources and detectors. Such movements may be curvilinear translations, rotations, revolutions, and/or reciprocations. The optical imaging system may also include a movable member into which all of the wave sources and detectors are incorporated. The actuator member may generate at least one another secondary movement of the movable member in addition to the primary movement of the wave sources and detectors.

In another aspect of the present invention, a method is provided to generate images of a target area of a physiological medium by an optical imaging system, where the images represent spatial or temporal distribution of chromophore properties of the medium. The optical imaging system includes at least one of the foregoing wave sources and at least one of the foregoing wave detectors, at least one movable member, and an actuator member. The movable member includes at least one of the wave source and detector, operationally couples with the actuator member, and forms at least one movable scanning unit having a longitudinal axis connecting the wave source and detector and defining a scanning area therearound. The actuator member generates movements of the movable member and/or scanning unit along at least one curvilinear path. The image generating method generally includes the steps of positioning the movable member on the target area, positioning the scanning unit in a first region of the target area, scanning the first region by irradiating electromagnetic waves into the medium by the wave source and obtaining the output signal from the medium by the wave detector, determining the chromophore property in the first region of the target area, and manipulating the actuator member to move the movable member and/or scanning unit from the first region to another region of the target area of the medium.

Embodiments of this aspect of the present invention may include one or more of the following features.

The foregoing image generating method includes the steps of repeating the above scanning, determining, and manipulating steps at different regions of the target area, and obtaining the images representing spatial distribution of the chromophore property in the target area. In the alternative, the scanning and determining steps may include the steps of scanning the target area over time, determining the chromophore property in the target area over time, and obtaining the images representing temporal variation of the property of the chromophore in the target area. The image generating method may include the steps of forming optical couplings between the target area of the medium and the sensors (i.e., wave source and detector) and maintaining such optical couplings during the movement of the movable member and/or scanning unit.

The manipulating step may include the step of moving the movable member at a constant speed or at speeds which vary with respect to time and/or position on the target area. The actuator member may move the movable member along the curvilinear path that may be substantially orthogonal to, parallel with or form a pre-determined angle with the longitudinal axis of the scanning unit. The manipulating step may also include the steps of linearly translating the movable member along a linear path, translating it along a curved path, rotating it about a center of rotation around a pre-selected angle, revolving it about a center of rotation for a pre-selected number of turns or reciprocating it along the same or different curvilinear path. The manipulating step may also include the steps of providing at least one guiding track along the target area or along a body of the optical imaging system and guiding the movable member therealong during such movement. In the alternative, the manipulating step may further include of the step of generating at least two movements of the movable member along at least one curvilinear path. The generating step may include the step of generating at least a portion of such first movement and at least a portion of such second movement simultaneously or sequentially.

The image generating method may include the steps of defining a first set of first voxels in the target area, determining a first sequence of first voxel values of the first voxels, where each first voxel value represents a first average of the chromophore property in an area or a volume of each first voxel, and generating the images of the distribution of the chromophore property directly from the foregoing first voxel values. The first voxel value (as well as second and third voxel values to be described below) is generally obtained from a set of solutions of the wave equations applied to the wave sources and detectors that define the particular voxel for such first voxel value. The generating step may also include the step of controlling image resolution by adjusting at least one dimension of such voxels, by adjusting, e.g., a distance between the wave source and detector, geometric arrangement therebetween, shape of the curvilinear path, length of the curvilinear path, tortuosity of the curvilinear path, number of movements of the movable member over different regions of the target area, speed of the movement of the movable member, and/or sampling rate (or data acquisition rate) of the output signal by the imaging member.

The image generating method may include the steps of defining a second set of second voxels in the target area, determining a second sequence of second voxel values of the second voxels, defining a first set of first cross-voxels where each first cross-voxel is an overlapping or intersecting portion of two or more intersecting voxels and where each intersecting voxel belongs to a different set of the first and second voxels and intersects the other in the overlapping portion, obtaining a first sequence of first cross-voxel values of the first cross-voxels where each first cross-voxel value is “directly” determined from the voxel values of the intersecting voxels, and generating the images representing the distribution of the chromophore property in the target area “directly” from the first cross-voxel values. The obtaining step may include the step of determining the foregoing first cross-voxel values by, e.g., arithmetically, geometrically, weight- or ensemble-averaging the voxel values of the intersecting voxels.

The image generating method may further include the steps of defining a third set of third voxels in the target area, determining a third sequence of third voxel values of the third voxels, defining a second set of second cross-voxels each of which is defined as an overlapping or intersecting portion of two or more intersecting voxels each belonging to a different set of voxels and intersecting the other in the overlapping portion, obtaining a second sequence of second cross-voxel values of the second cross-voxels where each second cross-voxel value is “directly” determined from the voxel values of the intersecting voxels, and generating the images of the distribution of the chromophore property “directly” from the second cross-voxel values. In addition, the first and second sequences of the first and second cross-voxel values may be combined to generate the images having an enhanced resolution.

In yet another aspect, a method is provided for generating images of a target area of a physiological medium by an optical imaging system including a sensor assembly comprised of at least one of the foregoing wave sources and at least one of the foregoing wave detectors, a body supporting at least a portion of the sensor assembly, and an actuator member operationally coupling with the sensor assembly and/or body and generating at least one movement of the sensor assembly and/or body. The image generating method includes the steps of positioning the sensor assembly in a first region of the target area, scanning the first region by the sensor assembly, determining the property of the chromophore in the first region, and manipulating the actuator member to generate movement of the sensor assembly and/or body from the first region toward another region of the target area along at least one curvilinear path.

Embodiments of this aspect of the present invention may include one or more of the following features.

The image generating method includes the steps of repeating such scanning, determining, and manipulating steps at multiple regions of the target area and obtaining the images representing spatial distribution of the chromophore property. In the alternative, the image generating method may include the steps of scanning the first region of the target area and determining the chromophore property in the first region over time, and obtaining such images representing temporal variation of the chromophore property in the target area.

The image generating method may include the steps of fixedly coupling the sensor assembly with the body and moving the body and sensor assembly simultaneously. Alternatively, the image generating method may include the steps of movably coupling the sensor assembly with the body and moving the sensor assembly with respect to the target area and body during the movement thereof. The actuator member may also move the body sequentially or simultaneously with the sensor assembly.

In a further aspect of the invention, a method is provided to generate images of chromophore property in a target area of a physiological medium. The image generating method includes the steps of disposing at least two wave sources substantially linearly along a straight line and aligning at least two wave detectors substantially linearly along the same straight line. Therefore, a scanning unit is defined around the wave sources and detectors and includes a longitudinal axis which substantially coincides with the straight line, thereby defining a scanning area which is substantially narrower than the target area.

Embodiments of this aspect of the present invention may include one or more of the following features.

The image generating method may include the steps of scanning one region of the target area, determining the chromophore property in such region, and generating at least one movement of the wave sources and/or detectors to move these sensors to another region of the target area without moving other components of the optical imaging system. In addition, the scanning and generating steps may be repeated at different regions of the target area so that the optical imaging system can scan multiple regions of the target area all of which have a total area substantially greater than the scanning area of the scanning unit and corresponding to a pre-selected portion of the target area.

Such steps may be terminated after a pre-selected number of repetitions or when the total area of the scanned regions reaches a pre-selected portion of the target area which may amount to a substantial or entire portion of the target area.

All wave sources may be aligned substantially linearly along the straight line in the positioning step. In the alternative, all wave detectors may be disposed substantially linearly and aligned along the same straight line in the same step.

The generating step may include the steps of coupling an actuator member with the wave sources and/or detectors, and manipulating the actuator member to generate the movement of some or all of these sensors along at least one curvilinear path without moving other components of the optical imaging system. Alternatively, the wave sources and detectors may be manually moved along at least one curvilinear path while maintaining other components of the optical imaging system in their positions. The wave sources and detectors may also be moved to and repositioned in other target areas of the physiological medium by a separate moving mechanism while maintaining the geometric arrangement therebetween.

In another aspect of the invention, a method is provided for calibrating an optical imaging system including at least one of the foregoing wave sources and at least one of the foregoing wave detectors. The calibrating method includes the steps of generating a first output signal from a first target area of the medium by the wave detector, identifying at least a portion of the first output signal which has a substantially flat profile and first similar magnitudes, obtaining a first baseline of the first output signal by obtaining a representative value of such first similar magnitudes, and non-dimensionalizing (i.e., normalizing) the first output signal by the first baseline to provide a self-calibrated first output signal.

Embodiments of this aspect of the present invention may include one or more of the following features.

The generating step includes the step of moving the wave sources and/or detectors across different regions of the first target area while the wave detectors generate the first output signals during the movement continuously, at pre-selected time intervals, and/or at pre-determined locations of the target area. The identifying step includes the step of reducing high-frequency noise from the first output signal before the identifying step by, e.g., arithmetically, geometrically, weight- or ensemble-averaging the first output signal. Alternatively, the high-frequency noise may be removed by processing at least a portion of the first output signal through a low-pass filter. The identifying step may also include the steps of selecting a threshold magnitude and identifying portion(s) of the first output signal that has magnitudes greater (or less) than the threshold magnitude which may be selected manually or may be determined adaptively based on the characteristics of the first output signal itself. For example, a reference magnitude may be identified by finding a local or global maximum (or minimum) magnitude of the first output signal and by obtaining the threshold magnitude therefrom, e.g., by multiplying a pre-selected factor to the reference magnitude or by substituting the reference magnitude into a semi-empirical or empirical equation to yield the threshold magnitude. The obtaining step may also include the step of arithmetically, geometrically, weight- or ensemble-averaging the first similar magnitudes of the flat portion(s) of the first output signal.

The normalizing step may include the steps of producing a difference signal which represents a difference between the first baseline and the first output signal and then dividing the difference signal by the first baseline of the first output signal to yield the self-calibrated first output signal.

The calibration method also includes the steps of moving the wave sources and/or detectors to a second region of the target area, generating a second output signal from the second region of the target area, and normalizing the second output signal by the first baseline to provide a self-calibrated second output signal. The generating and normalizing steps may be repeated in other regions of the target area as well. The calibration method may further include the steps of identifying at least a portion of the second output signal which is also substantially flat and has second similar magnitudes, and obtaining a second baseline of the second output signal which is a representative value of the second similar magnitudes. Thereafter, a representative baseline or a composite baseline may be calculated from the foregoing multiple baselines, e.g., by arithmetically, geometrically or weight-averaging such baselines or by manually selecting one of the baselines as the composite baseline.

In yet another aspect, a method is provided for obtaining a baseline of the foregoing optical imaging system including the foregoing wave sources and wave detectors. The calibration method includes the steps of generating a first output signal from a first target area, identifying from the first output signal at least one segment which is attributed to at least one homogeneous or normal region of the first target area, and obtaining a first baseline of the output signal which is a representative value of the segment attributed to the homogeneous or normal region of the first target area.

Embodiments of this aspect of the present invention may include one or more of the following features.

The generating step may include the steps of moving the wave sources or detectors across the first target area while generating the first output signal by the wave detectors during such movement. The foregoing segment is generally substantially flat and has similar magnitudes which may lie within a pre-selected range and/or which may vary no more than a pre-selected deviation throughout an entire length of the segment.

The obtaining step includes the step of arithmetically averaging the entire output signal when the entire target area is the normal region, arithmetically averaging the flat segment of the output signal when the target area includes the normal region as well as at least one abnormal region, or generating movement of the wave sources and/or detectors to a second target area when the entire first target area is the abnormal region.

In a further aspect of the invention, a method is provided for calibrating an optical imaging system with the foregoing wave sources and detectors on a real time basis. The calibration method includes the steps of positioning the wave sources and detectors in a first target area, generating a first output signal from the first target area, identifying at least a portion of the first output signal that is substantially flat and has first similar magnitudes, obtaining a baseline of the first output signal which is a representative value of the first similar magnitudes of the flat portion, and normalizing the first output signal by the baseline thereof within a pre-selected time interval and before the wave sources and/or detectors are moved to another target area of the medium. Accordingly, the foregoing optical imaging system can provide the self-calibrated output signals of multiple target areas of the medium on a substantially real time basis.

Embodiments of this aspect of the present invention may include one or more of the following features.

The calibration method may include the steps of displaying the first output signals, self-calibrated first output signals, distribution pattern of the first output signals, and distribution pattern of the self-calibrated first output signals, and moving the wave sources and/or detectors to a second target area of the medium. The generating step also includes the steps of moving the wave sources and/or detectors across different regions of the first target area, while the wave detectors generate the first output signals during such movement. The displaying step may include the steps of moving the wave sources and/or detectors across at least a substantial portion of the first target area and displaying the foregoing signals and/or distribution patterns without moving other components of the optical imaging system toward the adjacent target area.

In yet another aspect of the invention, a method is provided for generating images of a target area of a physiological medium by one of the foregoing optical imaging systems including the foregoing wave sources and detectors. The image generating method includes the steps of positioning or placing the foregoing movable member on the target area, positioning or placing the wave sources and detectors in a first region of the target area, generating a first movement of the wave sources and/or detectors over the target area along a first curvilinear path, defining a first set of first voxels during the first movement thereof while irradiating electromagnetic waves into the target area by the wave sources and generating the output signal from the target area by the wave detectors, determining a first sequence of first voxel values of the first voxels, each first voxel value representing an average of the chromophore property in each of the first voxels, generating a second movement of the wave sources and/or detectors over the substantially identical target area along a second curvilinear path, defining a second set of second voxels during such second movement while irradiating electromagnetic waves into the target area and generating the output signal therefrom, determining a second sequence of second voxel values of the second voxels where each second voxel value represents an average of the chromophore property in each of the second voxels, constructing a first set of first cross-voxels each of which is defined as an intersecting or overlapping portion of two or more intersecting voxels each of which belongs to a different set of voxels, each of which is defined along a different voxel axis, and each of which intersects the other at the first cross-voxel, calculating a first sequence of first cross-voxel values of the first cross-voxels, each first cross-voxel value determined “directly” from the voxel values of the intersecting voxels, and generating the images of the distribution of the chromophore property “directly” from the first sequence of the first cross-voxel values.

Embodiments of this aspect of the present invention may include one or more of the following features.

The defining steps may include the step of generating one or more of such voxels in a unit area or a unit volume of the target area on which the image generation is based. The defining steps may include the step of defining the first and/or second voxels per, e.g., each pre-selected distance along the curvilinear path of the movement, each pre-selected time interval during the movement of the foregoing movable member or each sampling interval of the output signal by the foregoing imaging member. The defining steps may further include the step of adjusting image resolution by varying at least one dimension of the foregoing voxels and/or cross-voxels.

The determining step may include the step of averaging the chromophore properties over an area or volume of the first and/or second voxels. The constructing step may include the step of generating the foregoing cross-voxels each of which represents the overlapping portion of the area- or volume-based intersecting voxels. The calculating step may include the step of arithmetically, geometrically, weight- or ensemble-averaging the first and second voxel values of the intersecting voxels.

In a further aspect, another method is provided for generating the images by one of the foregoing optical imaging systems. The image generating method includes the steps of placing the foregoing movable member on a target area of the medium, positioning the foregoing scanning unit in a first region of the target area, manipulating the foregoing actuator member to generate a first movement of the movable member from the first region to a second region of the target area along a first curvilinear path, defining a first set of first voxels along the first curvilinear path while irradiating electromagnetic waves thereinto and obtaining the output signal therefrom, determining a first sequence of first voxel values of the first voxels where each of the first voxel values represents an average of the property of the chromophore in each of the first voxels, manipulating the actuator member to generate a second movement of the movable member from a third region to a fourth region of the target area along a second curvilinear path, defining a second set of second voxels along the second curvilinear path while irradiating electromagnetic waves thereinto and obtaining the output signal therefrom, determining a second sequence of second voxel values of the second voxels, each second voxel value corresponding to an average of the chromophore property in each of the second voxels, constructing a first set of first cross-voxels each defined as an overlapping portion of two or more intersecting voxels each of which belongs to a different set of voxels, each of which is defined along a different curvilinear path of the movable member, and each of which intersects the other at each of the first cross-voxels, calculating a first sequence of first cross-voxel values of the first cross-voxels, where each of the first cross-voxel values is determined “directly” from the voxel values of two or more intersecting voxels, and generating the images of the chromophore properties or distribution thereof “directly” from the first sequence of the first cross-voxel values.

Each of the foregoing optical imaging systems and methods of the present invention may incorporate analytical and/or numerical solution schemes disclosed in the commonly assigned co-pending U.S. non-provisional patent application bearing Ser. No. 09/664,972, entitled “A system and Method for Absolute Oxygen Saturation” by Xuefeng Cheng, Xiaorong Xu, Shuoming Zhou, and Ming Wang which has been filed on Sep. 18, 2000 and which is incorporated herein in its entirety by reference (referred to as “the '972 application” hereinafter). Such optical imaging systems can calculate absolute value of concentration of oxygenated hemoglobin, [HbO], absolute value of concentration of deoxygenated hemoglobin, [Hb], absolute value of oxygen saturation, SO₂, and temporal changes in blood volume, by adopting any of the foregoing solution schemes disclosed in the co-pending '972 application. Accordingly, such optical imaging system can provide the foregoing images of the chromophore properties which may allow physicians to make direct diagnosis of target areas of the medium based on the “absolute” or “relative” values thereof. In addition, operational characteristics of the optical imaging systems and methods of the present invention incorporating the foregoing solution schemes disclosed in the above co-pending '972 application are only minimally affected by the number of wave sources and/or detectors incorporated therein and by geometric configuration therebetween. Accordingly, unless otherwise specified, the optical imaging systems of the present invention may include any number of wave sources and/or detectors arranged in any geometric arrangements.

As used herein, a “chromophore” may mean a substance in a physiological medium which exhibits at least minimum optical interaction with electromagnetic waves transmitting therethrough. Such chromophores may include solvents of a medium, solutes dissolved in the medium, and/or other substances included in such medium. Examples of such chromophores may include, but not limited to, oxygenated hemoglobin, deoxygenated hemoglobin, cytochromes, cytosomes, cytosols, enzymes, hormones, neurotransmitters, chemical or chemotransmitters, proteins, cholesterols, apoproteins, lipids, carbohydrates, blood cells, water, and other optical materials present in the animal or human cells, tissues or body fluid. “Chromophores” may also include extra-cellular substances which may be injected into the medium for therapeutic and/or imaging purposes or for creating interaction with electromagnetic waves. Typical examples of such “chromophores” may include, but not limited to, dyes, contrast agents, and other image-enhancing agents, each of which may be designed to exhibit optical interaction with electromagnetic waves having wavelengths in a specific range to be disclosed below.

“Hemoglobins” are oxygenated hemoglobin (i.e., HbO) and/or deoxygenated hemoglobin (i.e., Hb). Unless otherwise specified, “hemoglobins” refer to both oxygenated and deoxygenated hemoglobins. “Total hemoglobin” means the sum of the oxygenated and deoxygenated hemoglobins.

The term “electromagnetic waves” generally refer to acoustic waves, sound waves, near-infrared rays, infrared rays, visible lights, ultraviolet rays, lasers, and photons.

“Property” of the chromophores (or hemoglobins) may be intensive property such as their concentrations, a sum of such concentrations, a difference therebetween, and a ratio thereof. Such “property” may also be extensive property such as, e.g., volume, mass, weight, volumetric flow rate, and mass flow rate of the chromophores (or hemoglobins).

The term “value” is an absolute or relative value which represents spatial or temporal changes in the property of the chromophores (or hemoglobins).

“Distribution” means two-dimensional or three-dimensional distribution of the values of the chromophores (or hemoglobins) or their properties. “Distribution” may be measured or estimated in a spatial and/or temporal domain.

Unless otherwise defined, all technical and scientific terms used herein have the same meaning as commonly understood and/or used by one of ordinary skill in the art to which this invention belongs. Although methods and materials similar or equivalent to those described herein can be applied and/or used in the practice of or testing the present invention, suitable methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of an optical imaging system according to the present invention;

FIG. 2 is a cross-sectional top view of an exemplary scanning unit according to the present invention;

FIG. 3 is a cross-sectional top view of another exemplary scanning unit according to the present invention;

FIG. 4A is a schematic diagram of the scanning unit of FIG. 3 arranged for linear translation according to the present invention;

FIG. 4B is a schematic diagram of images obtained by the scanning unit of FIG. 4A according to the present invention;

FIG. 4C is an example of two-dimensional spatial distribution of an output signal generated by a wave detector of FIG. 4A according to the present invention;

FIG. 5 is another schematic diagram of the scanning unit of FIG. 3 arranged for rotation according to the present invention;

FIG. 6A is a schematic diagram of the scanning unit of FIG. 3 arranged for linear translation along the X-axis according to the present invention;

FIG. 6B is a schematic diagram of the scanning unit of FIG. 3 arranged for rotation according to the present invention;

FIG. 6C is a schematic diagram of the scanning unit of FIG. 3 arranged for linear translation along the Y-axis according to the present invention;

FIG. 6D is a schematic diagram of images obtained by the scanning unit of FIGS. 6A to 6C according to the present invention;

FIG. 7 is another schematic diagram of the scanning unit of FIG. 3 arranged for simultaneous X-translation and Y-reciprocation according to the present invention;

FIG. 8 is a cross-sectional top view of yet another exemplary scanning unit according to the present invention;

FIG. 9 is a schematic diagram of a mobile optical imaging system according to the present invention;

FIG. 10 is a schematic diagram of an exemplary optical imaging system according to the present invention;

FIGS. 11A and 11B are images of blood volume of normal and abnormal breast tissues, respectively, both of which are measured by the optical imaging system of FIG. 10 according to the present invention; and

FIGS. 12A and 12B are images of oxygen saturation of normal and abnormal breast tissues, respectively, both of which are measured by the optical imaging system of FIG. 10 according to the present invention.

### DETAILED DESCRIPTION OF THE INVENTION

The following description provides various optical imaging systems arranged to provide images of two- or three-dimensional spatial or temporal distribution of properties of chromophores in a physiological medium. More particularly, the following description provides preferred aspects and embodiments of the optical imaging systems and optical probes thereof including movable scanning units, self-calibration algorithms, and real-time image construction algorithms.

In one aspect of the present invention, an optical imaging system is provided to generate images of spatial distribution or temporal variation of one or more properties of chromophores in a physiological medium.

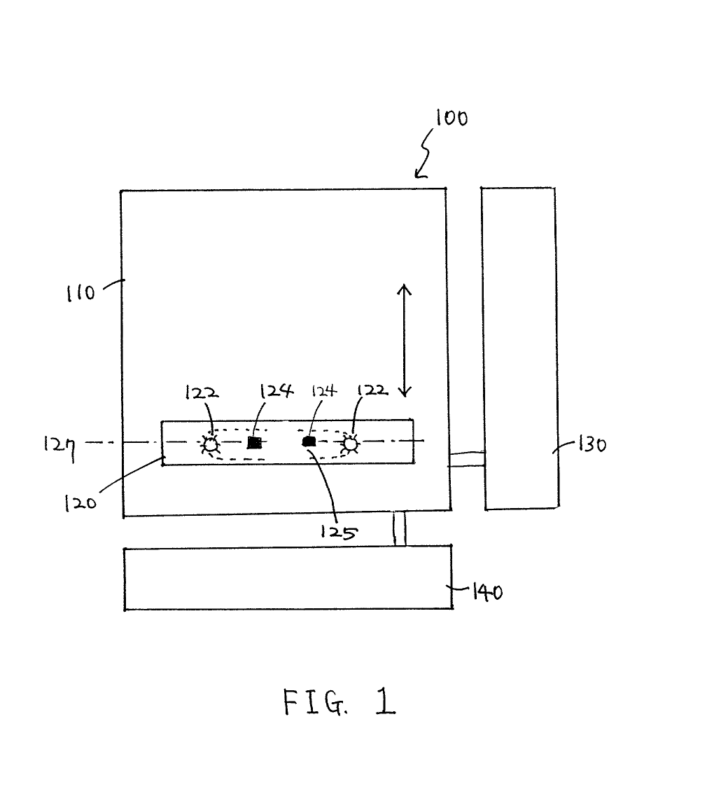

FIG. 1 is a schematic diagram of an optical imaging system according to the present invention. An exemplary optical imaging system 100 includes a body 110, movable member 120 having two wave sources 122 and two wave detectors 124, actuator member 130 arranged to move movable member 120 with respect to body 110 in the direction of the arrows, and imaging member 140 arranged to receive signals from the sensors (i.e., wave sources 122 and detectors 124) and to generate images of distribution of the property of the chromophore. By arranging a pre-determined number of wave sources 122 and detectors 124, they define a scanning unit 125 which forms a basic source-detector arrangement for scanning the medium.

Body 110 is generally made of rigid or semi-rigid material such as plastics. As will be explained below, shape and size of body 110 may be determined according to various design criteria which may include, e.g., an area of the medium to be scanned and examined (i.e., a “target area”), shape and size of movable member 120, characteristics of movements pf movable member 120, generated by actuator member 130, and configuration of curvilinear path of movable member 120.

Movable member 120 may include one or more wave sources, each arranged to form optical coupling with the medium and to irradiate electromagnetic waves thereinto. Any wave sources may be used in the movable member to irradiate electromagnetic waves having pre-selected wavelengths, e.g., in ranges between 100 nm and 5,000 nm, 300 nm and 3,000 nm or, more particularly, in the “near-infrared range” between 500 nm and 2,500 nm. As will be explained below, typical wave sources are arranged to irradiate the near-infrared electromagnetic waves having wavelengths of about 690 nm or about 830 nm. The wave sources may be arranged to emit electromagnetic waves with different wave characteristics such as, e.g., different wavelengths, phase angles, frequencies, amplitudes or harmonics. In the alternative, the wave sources may irradiate electromagnetic waves in which identical, similar or different signal waves are superposed to carrier electromagnetic waves having mutually distinguishable wavelengths, frequencies, phase angles, amplitudes or harmonics. The embodiment shown in FIG. 1 has an arrangement where movable member 120 includes two wave sources 122 each of which irradiates electromagnetic waves with different wave characteristics, e.g., wavelengths of about 680 nm to 700 nm and about 820 nm to 840 nm.

It is noted that the exact number of the wave sources included in the movable member is not critical in realizing the present invention which is described herein. For example, the movable member may include only a single wave source capable of irradiating multiple sets of electromagnetic waves having, e.g., different wave characteristics, identical or different signal waves or different or identical carrier waves, and so on. Such wave sources may be arranged to irradiate electromagnetic waves continuously, periodically or intermittently.

The movable member may include at least one wave detector arranged to detect electromagnetic waves and to generate output signal in response thereto. Any wave detectors may be used in this invention as long as they exhibit appropriate sensitivity to the electromagnetic waves having wavelengths in the foregoing ranges. The wave detectors may be arranged to detect multiple sets of electromagnetic waves each set of which may have foregoing different wave characteristics. The wave detectors may also be arranged to detect multiple sets of electromagnetic waves irradiated by multiple wave sources and to generate multiple sets of output signals accordingly. Alternatively, the movable member may also include a single wave detector which may be arranged to detect multiple sets of electromagnetic waves irradiated by multiple wave sources.

Movable member 120 may also include a scanning unit 125 extending along its longitudinal axis 127. Scanning unit 125 generally refers to a functional unit from which electromagnetic waves are irradiated into the medium and by which such electromagnetic waves interacted with the medium are detected. Accordingly, configuration of scanning unit 125 and its scanning area is predominantly determined by corresponding configuration of the sensor assembly and/or source-detector arrangement which is in turn determined by, e.g., number of wave sources or detectors, geometric arrangement therebetween, irradiation capacity or emission power of the wave sources, detection sensitivity of the wave detectors, etc. In the embodiment shown in FIG. 1, e.g., wave sources 122 and detectors 124 define substantially elongated scanning unit 125 where wave detectors 124 are interposed between two wave sources 122 along longitudinal axis 127 thereof. Although scanning unit 125 may be characterized by various dimensions (e.g., its length, width or height), a characteristic dimension of scanning unit 125 is generally the one which is orthogonal to its longitudinal axis 127. Thus, as will be explained in greater detail below, the characteristic dimension of scanning unit 125 of FIG. 1 is its width. It is appreciated that scanning unit 125 constitutes a portion of movable member 120 and that such scanning unit 125 preferably moves with movable member 120 by the actuator member 130. Therefore, unless otherwise specified, the terms “scanning unit” and “movable member” may be used herein interchangeably.

It is appreciated that the scanning unit may preferably define the scanning area which is continuous throughout an entire portion of the scanning unit so that a single measurement by the scanning unit generates the output signal covering the entire scanning area. For this purpose, the wave sources and detectors are preferably spaced at distances no greater than a threshold distance thereof. Selection of an optimal spacing between the wave sources and detectors is generally a matter of choice of one skilled in the art and may be determined by several factors which may include, but not limited to, optical properties of the physiological medium (e.g., absorption coefficient, scattering coefficient, and the like), irradiation capacity of the wave sources, detection sensitivity of the wave detectors, number of wave sources and/or detectors, geometric arrangement therebetween, and/or operational characteristics of the actuator member as will be explained below.

Actuator member 130 operationally couples with and generates movements of wave sources 122 and/or detectors 124 along at least one curvilinear path in at least one curvilinear direction. Any actuating devices may be incorporated into the optical imaging system for the purpose of generating foregoing movements. For example, a motor-gear assembly may be employed to generate rotations about a center of rotation around a pre-selected angle or to generate revolutions for a pre-selected number of turns. Alternatively, a stepper motor may be used, along with optional guiding tracks, to generate curvilinear translations, reciprocations, and combinations thereof, where examples of such curvilinear translations may be linear displacements along linear paths or non-linear translations along curved paths. The actuator member may also impart various temporal characteristics to such movements by generating, e.g., impulses (i.e., functions of δ(t)), steps (i.e., functions of u(t)), pulses, pulse trains, sinusoids, and combinations thereof. In addition, the actuator member may generate such movements continuously, periodically, and/or intermittently.

The actuator member may also generate at least two movements of the wave sources and/or detectors sequentially or simultaneously along at least two curvilinear paths in at least two curvilinear directions. Such movements may be along the curvilinear paths aligned to be substantially orthogonal to each other, as exemplified by the orthogonal axes of the Cartesian, cylindrical or spherical coordinate systems. Alternatively, the foregoing movements may take place along the identical or parallel curvilinear paths but in opposite directions, as exemplified in the reciprocating movements.

It is appreciated that the movable body, scanning unit, and actuator member may be arranged to provide various geometric arrangements between the longitudinal axis of the scanning unit and the curvilinear path of the movable member. For example, the scanning unit may be aligned with the actuator member in such a way that the scanning unit travels along its short axis which is orthogonal to the longitudinal axis of the scanning unit, rendering the curvilinear path of the scanning unit and/or movable member substantially orthogonal to the axis of the scanning unit. By the same token, the actuator member may move the scanning unit and/or movable member along the path substantially parallel with the axis of the scanning unit or along another path forming a pre-determined angle with the axis of the scanning unit.

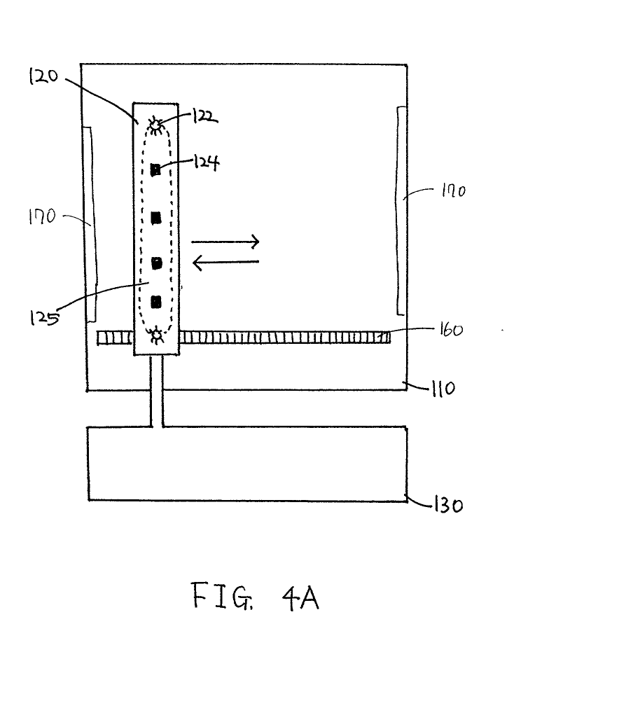

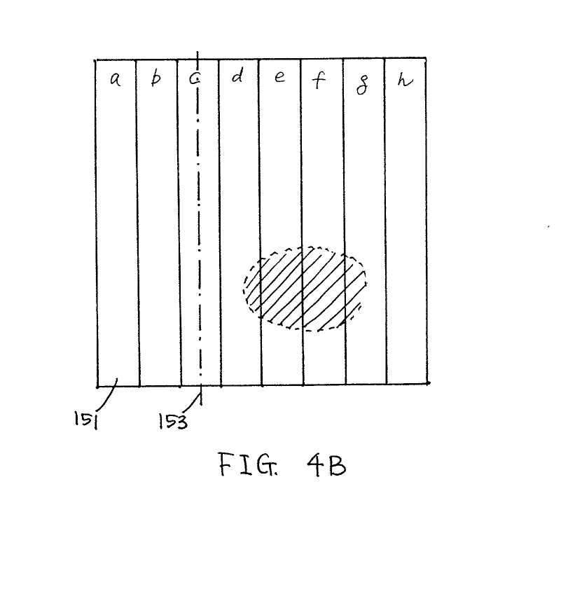

Furthermore, the actuator member may generate the foregoing movements at constant speeds or at speeds varying over time or position. An optional motion controller may be provided so that the speed of such movement may be controlled precisely according to a pre-determined pattern. Alternatively, such movement may also be controlled adaptive to various parameters such as, e.g., optical characteristics of the medium and/or presence or absence of abnormal regions in the target area which is signified by, e.g., abnormally high or low absorption or scattering of electromagnetic waves transmitted therethrough. Further details of the actuator member will be provided below in conjunction with the exemplary embodiments of the scanning units illustrated in FIGS. 4 through 10.

Imaging member 140 operationally couples with wave sources 122 and/or detectors 124 and is arranged to generate two- or three-dimensional images representing spatial and/or temporal distribution of the absolute or relative values of the chromophore properties in the medium. Imaging member 140 typically includes a data acquisition unit, algorithm unit, and image construction unit. The data acquisition unit receives and samples various signals which are to be used later by the algorithm unit to determine the absolute or relative chromophore properties. For example, the data acquisition unit may measure or receive signals related to intensity of electromagnetic waves irradiated by wave sources 122 and to such waves detected by wave detectors 124. The data acquisition unit may monitor other system variables or parameters related with the actuator member as well as an optional control member which may be arranged to control operation of each component of optical imaging system 100. The algorithm unit receives the foregoing signals or data from the data acquisition unit, solves multiple wave equations applied to wave sources 122 and detector 124, and obtains a set of solutions therefrom. Conventional analytical and/or numerical schemes may be incorporated into the algorithm unit to obtain solutions of such multiple wave equations, e.g., the photon diffusion equations, Beer-Lambert equations, modified Beer-Lambert equations, and their equivalents. The algorithm unit then determines the absolute or relative values of the chromophore properties directly from the solutions of the wave equations or by performing auxiliary calculations. The image construction unit is generally arranged to process or reorganize such absolute or relative values of the property of the chromophore and to provide images representing the two- and/or three-dimensional distribution of such property in the spatial and/or temporal domain.

The optical imaging systems of the present invention offer several benefits over prior art technologies such as conventional near-infrared spectroscopy, diffuse optical spectroscopy, etc. Conventional optical sensors generally define scanning units each of which allows only a single measurement in each measurement location. Therefore, when the target area is larger than the scanning area of such scanning unit, the sensor probe must be manually moved to different regions of the target area, and multiple measurements must be made thereat. Such procedure tends to lengthen the examination periods, not to mention unreliable images with poor resolution due to inconsistent positioning of the sensor probes on different measurement locations of the medium or due to inconsistent optical coupling formed at different measurement locations. Furthermore, conventional optical imaging technology requires a priori estimation of an baseline of output signals before scanning the target area of the medium. Considering the widely known fact that the baseline estimation constitutes a primary source of measurement errors, conventional optical imaging systems cannot be reliably used to obtain high-resolution images of a relatively large target area.

The optical imaging systems of the present invention can overcome such prior art deficiencies by, e.g., providing movable scanning units which can be positioned at one region (e.g., an edge) of the target area and sweep through different regions of a much larger target area without having to move and reposition other components of the system to other regions of the target area. Therefore, the foregoing optical imaging system can scan such large target area with the scanning unit forming the scanning area which amounts to only a fraction of the target area. The foregoing optical imaging systems also need fewer sensors (i.e., fewer wave sources or detectors) than their conventional counterparts. Thus, the optical probe of the present invention can be constructed as a light and compact article. In addition, by incorporating fewer sensors, noises attributed to idiosyncratic component variances inherent in each of the wave sources and detectors may also be reduced, thereby improving signal-to-noise ratios of the output signals and providing high-quality and high-resolution images therefrom. The optical imaging systems of the present invention may further be arranged to ensure that substantially identical optical couplings may be formed and maintained between the medium and movable wave sources and/or detectors during the movement of the movable member. As will be discussed below, this embodiment allows the foregoing optical imaging systems to establish a single baseline and to apply the same baseline to multiple output signals measured throughout different target areas of the entire medium. This embodiment further allows the use of a much simpler and more efficient image construction scheme capable of providing real-time images of the properties of the chromophores in the medium.

Though any analytical or numerical schemes may be used by the algorithm unit or image construction unit of the imaging member, an exemplary algorithm or image construction unit of the present invention preferably employs solution schemes disclosed in the co-pending '972 application. For example, the absolute values of concentration of deoxygenated hemoglobin, [Hb], concentration of oxygenated hemoglobin, [HbO], and oxygen saturation, SO₂, are obtained by the following equations (1a) to (1e) each of which corresponds, respectively, to the equations (8a) to (8d) and (9b) of the co-pending '972 application:

$$\begin{matrix}[ Hb ] = \frac{{ϵ}_{HbO}^{{λ}_{1}} \frac{{OD}^{{λ}_{1}}}{{F}^{{λ}_{1}}} - {ϵ}_{HbO}^{{λ}_{1}} \frac{{OD}^{{λ}_{2}}}{{F}^{{λ}_{2}}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(1a)} \\ [ HbO ] = \frac{{ϵ}_{Hb}^{{λ}_{1}} \frac{{OD}^{{λ}_{2}}}{{F}^{{λ}_{2}}} - {ϵ}_{Hb}^{{λ}_{2}} \frac{{OD}^{{λ}_{1}}}{{F}^{{λ}_{1}}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(1b)}\end{matrix}$$

F^{λ}^{₁}=(B_{S1D2}^{λ}^{₁}L_{S1D2}−B_{S1D1}^{λ}^{₁}L_{S1D1})+(B_{S2D1}^{λ}^{₁}L_{S2D1}−B_{S2D2}^{λ}^{₁}L_{S2D2}) (1c)

F^{λ}^{₂}=(B_{S1D2}^{λ}^{₂}L_{S1D2}−B_{S1D1}^{λ}^{₂}L_{S1D1})+(B_{S2D1}^{λ}^{₂}L_{S2D1}−B_{S2D2}^{λ}^{₂}L_{S2D2}) (1d)

$${SO}_{2} = \frac{{ϵ}_{Hb}^{{λ}_{1}} \frac{{OD}^{{λ}_{2}}}{{OD}^{{λ}_{1}}} \frac{{F}^{{λ}_{1}}}{{F}^{{λ}_{2}}} - {ϵ}_{Hb}^{{λ}_{2}}}{( {ϵ}_{Hb}^{{λ}_{1}} - {ϵ}_{HbO}^{{λ}_{1}} ) \frac{{OD}^{{λ}_{2}}}{{OD}^{{λ}_{1}}} \frac{{F}^{{λ}_{1}}}{{F}^{{λ}_{2}}} + ( {ϵ}_{HbO}^{{λ}_{2}} - {ϵ}_{Hb}^{{λ}_{2}} )} \qquad \text{(1e)}$$

where the parameters “ε_{Hb}” and “ε_{HbO}” represent extinction coefficients of the deoxygenated and oxygenated hemoglobins, respectively, the variable “OD” is an optical density defined as a logarithmic ratio of light intensities (i.e., magnitudes or amplitudes of electromagnetic waves) detected by a wave detector, the parameter “B” is conventionally known as a path length factor, the parameter “L_{SiDj}” is a distance between the i-th wave source and j-th wave detector, and the superscripts “λ₁” and “λ₂” represent that a system parameter or variable is obtained by irradiating electromagnetic waves having wavelengths λ₁ and λ₂, respectively.

Alternatively, the algorithm unit or image construction unit of the imaging member may employ the over-determined iterative method as disclosed in the foregoing '972 application, where the absolute values of [Hb], [HbO], and SO₂ are determined by the following equations (2a) to (2c), each of which corresponds to the equations (17a) through (17c) of the co-pending '972 application, respectively:

$$\begin{matrix}[ Hb ] = \frac{{ϵ}_{HbO}^{{λ}_{2}} {μ}_{a}^{{λ}_{1}} - {ϵ}_{HbO}^{{λ}_{1}} {μ}_{a}^{{λ}_{2}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(2a)} \\ [ HbO ] = \frac{{ϵ}_{Hb}^{{λ}_{1}} {μ}_{a}^{{λ}_{2}} - {ϵ}_{Hb}^{{λ}_{2}} {μ}_{a}^{{λ}_{1}}}{{ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}}} & \text{(2b)} \\ \begin{matrix}{SO}_{2} = \frac{[ HbO ]}{[ Hb ] + [ HbO ]} \\ = \frac{{ϵ}_{Hb}^{{λ}_{1}} {μ}_{a}^{{λ}_{2}} - {ϵ}_{Hb}^{{λ}_{2}} {μ}_{a}^{{λ}_{1}}}{( {ϵ}_{HbO}^{{λ}_{2}} {μ}_{a}^{{λ}_{1}} - {ϵ}_{HbO}^{{λ}_{1}} {μ}_{a}^{{λ}_{2}} ) + ( {ϵ}_{Hb}^{{λ}_{1}} {μ}_{a}^{{λ}_{2}} - {ϵ}_{Hb}^{{λ}_{2}} {μ}_{a}^{{λ}_{1}} )}\end{matrix} & \text{(2c)}\end{matrix}$$

where the parameter “μₐ” denotes an absorption coefficient of the medium. It is noted that the imaging member of the present invention may be arranged to receive the output signals generated by the wave detectors and to calculate optical densities which may be supplied to the algorithm unit or image construction unit. Once the absolute values of or their changes in the concentrations of the hemoglobins are determined, the imaging member generates images representing two- or three-dimensional spatial and/or temporal distributions of the hemoglobins by employing a real-time image construction technique as will be discussed in greater detail below.

In the alternative, changes in the hemoglobins distribution are determined by estimating changes in optical characteristics of the target area of the medium. For example, changes in concentrations of oxygenated and deoxygenated hemoglobins may be calculated from the differences in their extinction coefficients which are measured by electromagnetic waves having two different wavelengths. In an exemplary numerical scheme, the photon diffusion equations may be modified and solved by applying the diffusion approximation described in, e.g., Keijer et al., “Optical Diffusion in Layered Media,” Applied Optics, vol. 27, p. 1820-1824 (1988) and Haskell et al., “Boundary Conditions for Diffusion Equation in Radiative Transfer,” Journal of Optical Society of America, A, vol. 11, p. 2727-2741, 1994:

$${[ \begin{matrix}{Φ}_{SC} ( {r}_{S1} , {r}_{D1} ) \\ ⋮ \\ {Φ}_{SC} ( {r}_{SM} , {r}_{DM} )\end{matrix} ]}_{M , 1} = {[ \begin{matrix}{W}_{11} & ⋯ & {W}_{1 N} \\ ⋮ & ⋰ & ⋮ \\ {W}_{M1} & ⋯ & {W}_{MN}\end{matrix} ]}_{M , N} \cdot {[ \begin{matrix}{Δμ}_{a , 1} \\ ⋮ \\ {Δμ}_{a , N}\end{matrix} ]}_{N , 1} \qquad ( 3 )$$

where the symbol “Φ_{SC}(r_{Si}, r_{Dj})” represents a normalized optical density measured by a j-th wave detector in response to an i-th wave source, the variables “r_{Si}” and “r_{Dj}” are positions of the i-th wave source and j-th wave detector, respectively, the symbol “Δμ_{a,1}” denotes tissue optical perturbation such as the changes in the absorption coefficient in an i-th voxel, the parameters “M” and “N” are the number of measurements and the voxel number to be reconstructed, respectively, and the variable “Wᵢⱼ” is a weight function which represents the probability that a photon travels from the i-th wave source to a certain point inside the target area of the medium and is then detected by the j-th wave detector. The weight function, Wᵢⱼ, of equation (3) is defined as:

$${W}_{ij} = \frac{G ( {r}_{Di} , {r}_{j} ) \cdot {Φ}_{0} ( {r}_{Si} , {r}_{j} ) \cdot v \cdot {h}^{3}}{{D}_{photon}} \qquad ( 4 )$$

where the parameters “h³” is the volume of a voxel, “Dₚₕₒₜₒₙ” represents a photon diffusion coefficient, and “ν” denotes the velocity of light in the physiological medium. In addition, the variable “Φ_{SC}(r_{Si}, r_{Dj})” is the normalized optical density which is defined as:

$${Φ}_{SC} ( {r}_{si} , {r}_{{D}_{j}} ) = \frac{{I}_{B} - I}{{I}_{B}} \qquad ( 5 )$$

where the variable “I” represents the output signal measured by the sensor assembly which is comprised of the i-th wave source and j-th wave detector disposed at positions “r_{Si}” and “r_{Dj},” respectively, and the variable “I_{B}” denotes a baseline of the output signal determined by the wave detector.

Various methods such as, e.g., the direct matrix inversion and simultaneous iterative reconstruction techniques, may be applied to solve the above set of equations (3) to (5). Once the tissue optical perturbations, “Δμₐ^{λ}^{₁}” and “Δμₐ^{λ}^{₂}” are estimated by irradiating electromagnetic waves having two different wavelengths, λ₁ and λ₂, respectively, changes in concentrations of oxygenated hemoglobin and deoxygenated hemoglobin can be obtained as follows:

$$\begin{matrix}Δ [ Hb ] = \frac{{ϵ}_{HbO}^{{λ}_{2}} \cdot {Δμ}_{a}^{{λ}_{1}} - {ϵ}_{HbO}^{{λ}_{1}} \cdot {Δμ}_{a}^{{λ}_{2}}}{( {ϵ}_{Hb}^{{λ}_{1}} {ϵ}_{HbO}^{{λ}_{2}} - {ϵ}_{HbO}^{{λ}_{1}} {ϵ}_{Hb}^{{λ}_{2}} ) \cdot L} & \text{(6a)} \\ Δ [ HbO ] = \frac{{ϵ}_{Hb}^{{λ}_{1}} \cdot {Δμ}_{a}^{{λ}_{2}} - {ϵ}_{Hb}^{{λ}_{2}} \cdot {Δμ}_{a}^{{λ}_{1}}}{( {ϵ}_{Hb}^{{λ}_{1}} {ϵ}_{HbO}^{{λ}_{2}} - {ϵ}_{Hb}^{{λ}_{2}} {ϵ}_{HbO}^{{λ}_{1}} ) \cdot L} & \text{(6b)}\end{matrix}$$

where L is the distance between the wave source and detector and the parameters ε_{Hb}^{λ}^{₁}, ε_{Hb}^{λ}^{₂}, ε_{HbO}^{λ}^{₁}, and ε_{HbO}^{λ}^{₂} are the extinction coefficients of oxygenated hemoglobin and deoxygenated hemoglobin at two different wavelengths, λ₁ and λ₂, respectively.

Incorporating any of the foregoing solution schemes of the '972 application into the optical imaging systems of the present invention offers additional benefits over the prior art optical imaging technology. Contrary to the CWS which allows measurement of changes in the chromophore concentrations, the foregoing optical imaging systems provide a direct means for assessing spatial distribution or temporal variation of the absolute values of the properties of the chromophores of the physiological medium, thereby allowing the physicians to make direct diagnosis based on such absolute values of the chromophore properties. Furthermore, as will be discussed in greater detail below, the foregoing optical imaging systems can readily be incorporated into any conventional optical imaging systems and their optical probes which may include any number of wave sources and/or detectors arranged in almost any arbitrary configurations. Therefore, the embodiments of the present invention discussed herein may be readily applied to construct optical imaging systems that can be customized to specific clinical applications without compromising their performance characteristics.

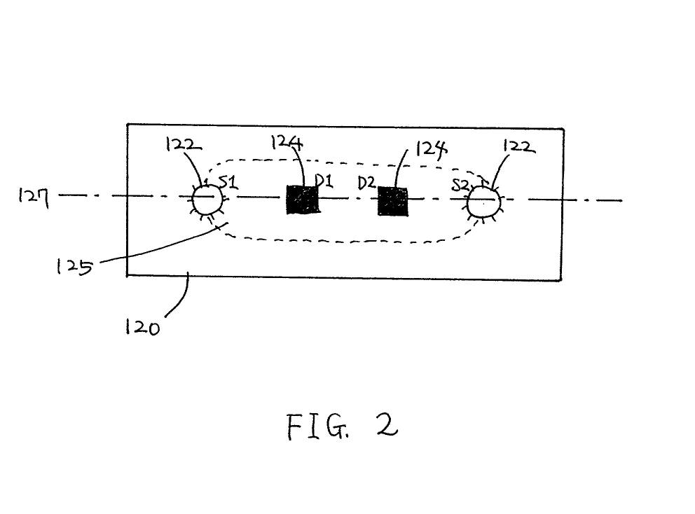

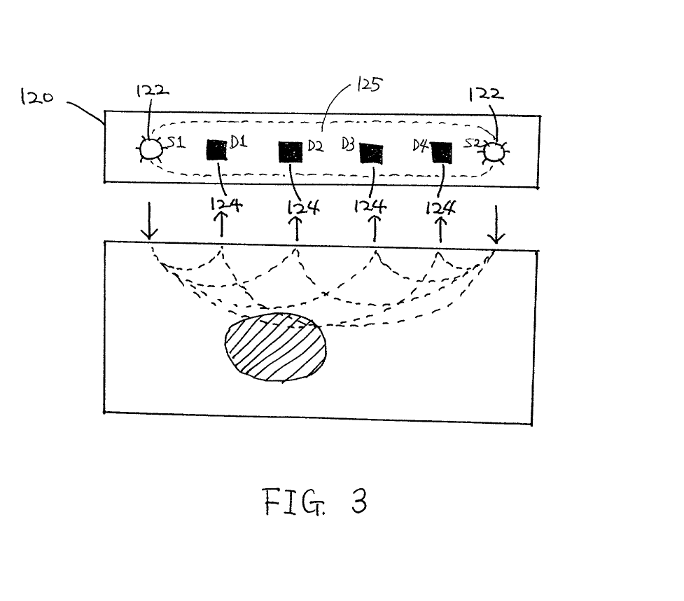

The optical imaging system of the present invention preferably determines the absolute or relative values of the chromophore properties by obtaining solutions of multiple wave equations by using one of the solution schemes disclosed in the co-pending '972 application. Accordingly, as far as the symmetry requirements of the '972 application are satisfied, operational characteristics of such optical imaging systems are generally not affected by actual configuration of the wave sources and/or detectors. Thus, the optical imaging system of the present invention preferably includes any number of wave sources and/or detectors arranged in almost any configurations, subject to the foregoing symmetry requirements. However, the sensor assembly or scanning unit of the present invention may preferably be constructed according to a few semi-empirical rules which are expected to provide enhanced accuracy, reliability, and/or reproducibility of the estimated absolute or relative values of the chromophore properties. Such exemplary design rules are: (1) each scanning unit preferably includes at least two wave sources and at least two wave detectors; and (2) the distances between the wave source and detector may not exceed a threshold sensitivity range of the wave detector which may range from, e.g., several to 10 cm or, in particular, about 5 cm for most human and animal tissues. FIGS. 2 and 3 describe a few exemplary embodiments of the scanning units constructed according to the foregoing design rules.

FIG. 2 is a cross-sectional top view of an exemplary movable member and scanning unit thereof according to the present invention. Contrary to conventional source-detector arrangements where each wave source is surrounded by multiple wave detectors or vice versa, scanning unit 125 of FIG. 2 is defined by two wave sources 122 (i.e., S1 and S2) each of which is disposed along longitudinal axis 127 thereof. Scanning unit 125 further includes two wave detectors 124 (i.e., D1 and D2) which are interposed between two wave sources 122 along the same axis 127 and spaced at substantially equal distances therefrom. Therefore, scanning unit 125 defines the scanning area elongated along the same axis 127 and having a characteristic width which may be determined by, e.g., irradiation capacity or emission power of wave sources 122, sensitivity or detection range of wave detectors 124, optical characteristics of the medium, and the like.

It is appreciated that scanning unit 125 of FIG. 2 does satisfy the symmetry requirements of the co-pending '972 application, i.e., the wave sources and detectors are arranged to maintain substantially identical near- and far-distances therebetween during the movement of the movable member and/or scanning unit. For example, a first near-distance between the wave source S1 and wave detector D1 is substantially similar or identical to a second near-distance between the wave source S2 and wave detector D2. In addition, a first far-distance between the wave source S1 and wave detector D2 is substantially similar or identical to a second far-distance between the wave source S2 and wave detector D1. The major advantage of this symmetric arrangement lies in the fact that electromagnetic waves are substantially uniformly transmitted, absorbed, and/or scattered throughout the entire area or volume of the target area of the medium. Accordingly, such scanning unit can provide uniform coverage of the target area and, therefore, improve accuracy and reliability of the output signals (e.g., improved signal-to-noise ratios thereof), and enhance the resolution of the images constructed therefrom.

It is also appreciated that scanning unit 125 of FIG. 2 has the source-detector arrangement which is substantially contrary to the general norms for constructing optical probes of the conventional optical imaging equipment. For example, conventional optical probes generally include a large number of wave sources and detectors which are distributed uniformly over a two-dimensional scanning field. Accordingly, the area of the medium that can be scanned thereby in a single measurement is at best as large as to the scanning field of the probes. To the contrary, the optical imaging system of the present invention includes significantly fewer wave sources and detectors which are aligned substantially along an axis of the movable member in a substantially one-dimensional fashion. This linear arrangement would be a fatal drawback for the conventional probes, because the scanning area defined by the linearly aligned sensors may only amount to a narrow strip. However, by arranging the actuator member to generate various movements of the scanning unit, the foregoing optical imaging system can cover the target area which may be significantly larger than the scanning area. Further benefits and advantages of such embodiment will be discussed in greater detail below.

FIG. 3 is a cross-sectional top view of another exemplary movable member and its scanning unit according to the present invention. Scanning unit 125 includes two wave sources 122 (S1 and S2) disposed along longitudinal axis 127 thereof and four wave detectors 124 (D1 to D4) each of which is interposed between two wave sources 122 and aligned along the same axis 127 at substantially equal distances. The embodiment of FIG. 3 is different from that of FIG. 2 in a few aspects. First of all, it is manifest that scanning unit 125 of FIG. 3 does not necessarily satisfy the near- and far-distance configuration of FIG. 2. For example, although the first and fourth wave detectors (D1 and D4) and the second and third wave detectors (D2 and D3) satisfy the symmetric requirements disclosed in the co-pending '972 application, the near- and far-distances are different for the first and third wave detectors (D1 and D3) and for the second and fourth wave detectors (D2 and D4). In addition, the banana-shaped paths (see the figure) of electromagnetic waves also reveal that each pair of wave source 122 and detector 124 covers different portions of the target area and, therefore, generates the output signals by detecting electromagnetic waves absorbed and scattered in different extent through different portions of the medium. However, by interposing all four wave detectors 122 between two wave sources 122 at equal distances, the entire target area of the medium may be substantially uniformly covered by the source-delector assembly of FIG. 3 along the thickness and/or depth of the medium. Accordingly, scanning unit 125 of FIG. 3 can also provide relatively uniform coverage of the medium throughout the entire scanning area or scanning volume. In addition, scanning unit 125 of FIG. 3 can provide a longer scanning area because it includes more wave detectors 124 and, therefore, can extend farther along axis 127 than the one in FIG. 2. Thus, an abnormality in the medium may be more easily detected with such longer scanning unit 125 by, e.g., comparing the output signals generated by wave detectors 124 that can cover the longer and possibly wider scanning area. As an example, a sudden increase or reduction in the output signals may imply that an abnormality such as a tumor having greater or less extinction or absorption coefficients may exist along the elongated scanning area or volume defined by a pair of the wave source and detector responsible for that curved output signal. Furthermore, because the output signals generated by scanning unit 125 cover a longer and possibly wider scanning area, imaging member 140 can provide a more reliable baseline of the output signal and, therefore, perform more accurate self-calibration of the output signals. Details of such self-calibration procedure will be provided below, e.g., in conjunction with FIG. 4C.

The source-detector arrangement may also be modified to provide scanning units having different configurations without departing from the scope of the invention. For example, the scanning unit may include three or more wave sources (or detectors), where at least two or all of wave sources (or detectors) may be disposed substantially linearly along the longitudinal axis of the scanning unit. The wave detectors (or sources) may further be interposed between two or more wave sources (or detectors) along the same axis of the scanning unit. Alternatively, the scanning unit may include at least two wave sources (or detectors), where the first wave source (or detector) is disposed on one side across the axis of the scanning unit, while the second wave source (or detector) is disposed on the other side across the axis. Such wave sources (or detectors) may be disposed symmetrically with respect to the axis of the scanning unit or with respect to a point of symmetry disposed in the scanning unit as well.

In another aspect of the present invention, an optical imaging system may include at least one wave source and at least one wave detector, each of which couples with a body which may be arranged stationary or mobile. Such optical imaging system may be arranged substantially similar to those of FIG. 1, e.g., including the foregoing body, at least one sensor assembly (corresponding to movable member 120 of FIG. 1) having at least one wave source and at least one wave detector, an actuator member for generating at least one of the foregoing movements of at least one of the body and movable member relative to the target area, and an imaging member for receiving signals from the sensor assembly and for generating the images of the chromophore property and/or distribution thereof.

In one embodiment, the body is arranged to be movable with respect to the target area, while the sensor assembly is fixedly coupled to a scanning surface of the body. Because the wave source and detector are fixedly coupled with the body and maintains a constant geometric arrangement therebetween, the actuator member moves the body so that a single movement of the body results in the movement of the sensor assembly and body in unison. This embodiment is useful for its simple mechanical construction and enhanced mechanical support attained by the fixed coupling between the sensor assembly and body.

In another embodiment, the actuator member generates separate movements of the sensor assembly and the body so that each of the sensor assembly and the body can move with respect to the other while moving itself with respect to the target area as well. Despite complicated design and control requirements, this embodiment is advantageous in providing the sensor assembly with greater flexibility in scanning different regions of the target area along the meticulous movement paths of the sensor assembly and/or the body.

Other embodiments pertaining to the foregoing optical imaging systems may also be applied to this aspect of the present invention of FIG. 2. For example, the actuator member may generate one or more movements continuously, intermittently or periodically. The actuator member may also generate such movement at constant speeds or at speeds varying over time and/or position. In the alternative, the actuator member may further be arranged to generate such movements simultaneously or sequentially.

In yet another aspect of the invention, an optical imaging system includes at least one of the foregoing wave sources, at least one of the foregoing wave detectors, an actuator member, at least one optical probe including a movable member, a console (or main body), and a connector member. In general, the actuator member generates at least one movement of the wave source, wave detector, and/or movable member along at least one curvilinear path. The connector member provides various communications between the optical probe and console. For example, the connector member may include power lines and/or electrical wire to deliver electric power and/or to transmit analog or digital data. The connector member may include optical pathways such as fiber optic products to transmit electromagnetic waves or optical signals between the probe and console. Furthermore, the connector member may provide mechanical support between the probe and the console or transmit translating, rotating, revolving or reciprocating power generated by the actuator member to the movable member through power transmission pathways such as a flexible power cable or universal joint.

In one embodiment, the movable member of the optical probe includes at least one of the wave source and detector. Electric power may be supplied by an internal power mechanism of the optical probe or from the console through the connector member. The actuator member may be disposed in the optical probe to move at least one of the wave source and detector, or may be disposed in the console where the translational, rotational, revolving or reciprocating power may be transmitted to the movable member through the connector member. Similarly, the imaging member may be disposed at either of the optical probe and console.

In another embodiment, the console may include at least one of the wave source and at least one of the wave detectors. The movable member of the optical probe includes minimum instrumentation only to the extent that the movable member receives electromagnetic waves from the wave source of the console and transmits such waves into the target area of the medium and that the movable member detects the electromagnetic waves from the target area and transmits the foregoing waves toward the wave detector of the console. In one exemplary embodiment, the movable member may define two apertures on its scanning surface. A first optical fiber is disposed between the wave source and the first aperture, and the second optical fiber is disposed between the wave detector and the second aperture. By arranging the first and second apertures to form appropriate optical couplings with the medium, the target area may be indirectly scanned by the wave source and wave detector through the optical pathways of the connector member. Similar to the foregoing embodiment, electric power may be supplied to the optical probe by its own internal power mechanism or from an external or main power mechanism of the console through the connector member. The actuator member may be disposed in the optical probe to move at least one of the first and second apertures over different regions of the target area or different target areas of the physiological medium. Alternatively, the actuator member may be disposed in the console so that translational, rotational, revolving or reciprocating power generated thereby may be mechanically transmitted to the movable member through the connector member. Similarly, the imaging member may be disposed at either of the optical probe and console.

In any of the foregoing embodiments, an optional screen may be provided to the optical probe so as to allow an operator to view raw images (e.g., images of distribution patterns of system variables such as the output signals generated by the wave detector), processed images (e.g., images of distribution patterns of functions or solutions obtained by processing the raw signal), and/or final images (e.g., images of the chromophore property and its distribution). Alternatively, when the imaging member is disposed at the console, the optical probe may include a data transmission unit to transmit the data to the imaging member on a real time, intermittent or periodic basis. The optical probe may also include a memory unit or storage member to temporarily or permanently store various signals.

The foregoing embodiments of this aspect of the invention offer benefits over the prior art technologies. First of all, bulky or heavy components such as a power supply, wave generator (such as a lamp, laser source or drive, and the like), photo-detector, detector drive, and/or circuit boards, may be included in the console, while only essential elements (e.g., optical apertures and optical fibers) are disposed in the portable probe. Thus, the movable member can maintain a compact size and light weight. Secondly, because the foregoing optical probes need fewer components, idiosyncratic errors due to component variances may also be minimized. Thirdly, the foregoing optical probe may be constructed as a semi-portable article wearable by a patient for continuous or periodic monitoring and imaging of the chromophore properties of the target area of the patient.

In a further aspect of the invention, an optical imaging system includes at least one portable probe and a console (or main body). The portable probe includes at least one movable member and an actuator member both of which are identical or substantially similar to those described hereinabove. For example, the movable member includes at least one of the foregoing wave sources and detectors, and the actuator member generates at least one movement of the movable member along at least one curvilinear direction. The console is generally arranged to include at least a portion of the imaging member.

In one embodiment, the portable probe and console operationally connect to each other via a connector member providing the foregoing communications therebetween. In another embodiment, the portable probe may be provided as a separate article which is physically detachable from the console. Such portable probe preferably includes at least one wave source, at least one wave detector, an actuator member such as a miniature motor assembly, and an internal power mechanism capable of supplying electric power to the above components of the portable probe. In addition, the portable probe may include either a data storage unit or data transmission unit so that the data may be temporarily stored or telemetrically transmitted to the console. The internal power mechanism may preferably be rechargeable and capable of sustaining operation of the portable probe for a pre-determined period. The primary advantage of this embodiment lies in the fact that such portable probe can be worn by a patient or even be implanted inside the patient for constant or periodic monitoring and/or imaging of various target areas.

In yet another aspect of the invention, an optical imaging system may include two or more wave sources and two or more wave detectors, where at least two of the wave sources and at least two of the wave detectors are disposed substantially linearly along a line which passes through, e.g., each of the wave sources and detectors.

It is noted that the linear arrangement of the wave sources and detectors generally results in the scanning unit substantially elongated along the line and having the scanning area which is also elongated and which is much narrower than the target area. By allowing the actuator member to generate the foregoing movements of the wave sources and/or detectors, the optical imaging system of the present invention enables the smaller scanning unit thereof to scan the entire target area.

The foregoing aspect of the invention offers numerous additional benefits. Prior art optical imaging machines typically rely on a single, large probe designed to cover the target area. Accordingly, the prior art probe has to include a large number of wave sources and detectors distributed on its sensing surface. By incorporating a large number of wave sources and detectors, the prior art technology suffers from various disadvantages. For example, such probe is generally big and bulky. Thus, unless the probe is arranged to conform to the curvature of the target area, some wave sources and/or detectors may be subject to poor optical coupling with the contoured target area. Even if such probe may be provided with a conforming surface, such target-specific probe may find limited utility. In addition, the output signals and final images generated thereby may include a significant amount of noise attributed to the idiosyncratic component variances among the sensors. To the contrary, the optical imaging system of the present invention typically defines the scanning unit comprising fewer sensors many or all of which may be linearly aligned along the longitudinal axis of the scanning unit. Therefore, the scanning unit shaped as a narrow sensor strip can more easily conform to the contour of the target area. By arranging the actuator member to translate and/or rotate the scanning unit to the different regions of the target area, the foregoing optical imaging system may scan the entire scanning area with a much smaller scanning unit while maintaining excellent optical couplings with the target area. The foregoing optical imaging system also requires fewer wave sources or detectors, thereby reducing manufacturing cost and thereby minimizing the noises attributed to the idiosyncratic component variances.

As discussed hereinabove, actuator members generate movements of the scanning unit to cover the target area of the medium which is substantially larger than the scanning area of the scanning unit. Following figures illustrate typical arrangements of the actuator member designed to generate various movements of the movable member. For the illustration purposes, the embodiment shown in FIG. 3 has been selected as the exemplary scanning unit throughout FIGS. 4 through 7.

FIG. 4A is a schematic diagram of the scanning unit of FIG. 3 arranged for linear translations according to the present invention. As described hereinabove, movable member 120 includes two wave sources 122 and four equi-spaced wave detectors 124 that are interposed between wave sources 122. Thus, scanning unit 125 is defined to have a substantially elongated shape and to extend along longitudinal axis 127 thereof. Stationary body 110 is preferably sized to be slightly larger than the desired target area of the medium to ensure body 110 to cover the entire target area. In this embodiment, body 110 has a rectangular (or square) shape to accommodate positioning and movement of elongated scanning unit 125. Actuating member 130 such as a stepper motor assembly linearly translates scanning unit 125 along a linear path which is aligned to be substantially parallel with an upper and lower sides of rectangular (or square) body 100. It is noted that at least a portion of body 110 may form a dead area or blind spot where scanning unit 125 cannot make any reliable measurements. Such dead area is generally confined to portions adjacent to comers or edges of body 110. The size (or width)of the dead area may depend on, e.g., a distance between an edge of body 110 and wave sources 122. Because the dead area generally wastes valuable real estate of body 110, it is preferably minimized by conforming the shape of body 110 to the size and shape of scanning unit 125 as well as to the curvilinear paths of the movements of scanning unit 125.

To generate high-precision movements of the scanning unit, the stationary body 110 may include one or more guiding tracks 160 which define the path of the linear translation. Alternatively, stationary body 110 maybe provided with barriers 170 along the edges thereof so that movements of scanning unit 125 may be confined inside the region bordered by such barriers 170 and that positioning or movement of the scanning unit beyond barriers 170 may be prevented.

The actuator member linearly translates the scanning unit at a pre-selected speed of translation. Alternatively, the actuating member may be provided with a control feature so that a user may manipulate the scanning unit to move at an appropriate speed, to move along a desired guiding track, and/or to have a recess between different movements of the scanning unit along different curvilinear paths. It is appreciated that, other factors being equal, the speed of the scanning unit generally adversely affects accuracy of the estimated values of the chromophore properties as well as the resolution of the final images thereof. Accordingly, the actuating member may be arranged to allow an operator to select an optimal speed of the scanning unit which may be determined based on several factors including, but not limited to, configuration of the scanning unit and/or movable member, desirable resolution of the final images, frequency responses of each component of the optical imaging system, and the like.

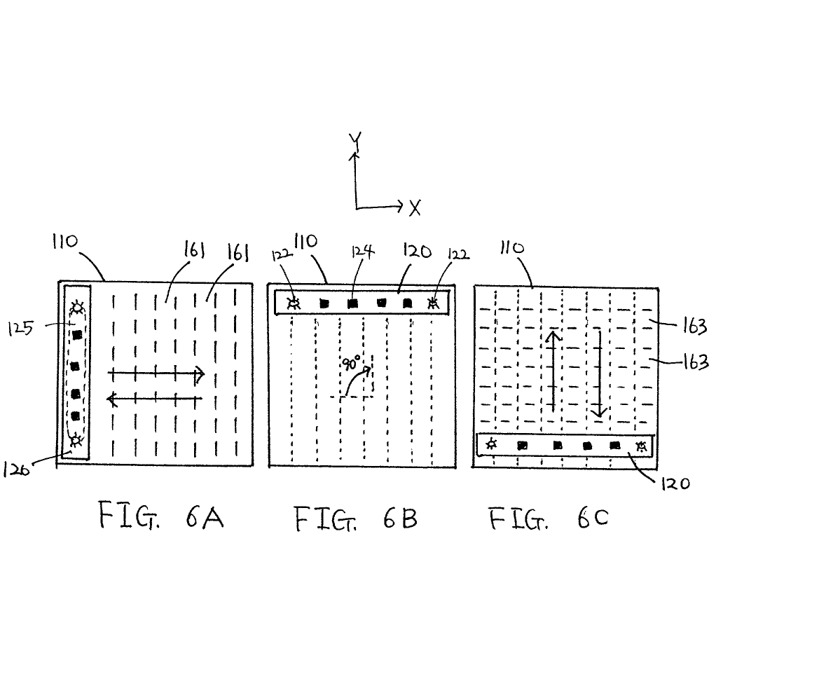

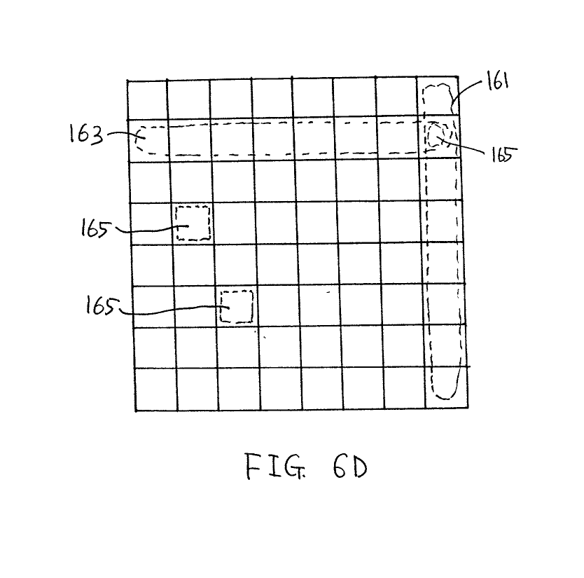

In operation, movable member 120 is placed on a desired target area of the medium and scanning unit 125 is positioned in a first region of the target area which is generally adjacent to one side of the rectangular target area so that wave sources 122 and detectors 124 can form optical couplings with the first region of the target area. Actuator member 130 is activated to linearly translate scanning unit 125 away from the first region toward a second region of the target area such as an adjacent or opposing side of the rectangular target area. Wave sources 122 and detectors 124 are manipulated to maintain the optical couplings with the medium during the linear translation of scanning unit 125 so that wave detectors 124 can generate the output signal during the translation. The imaging member receives and samples the output signal as well as other signals representing the system variables or parameters. The imaging member removes high-frequency noise from the output signals and determines a sequence of representative values of the chromophore property for a set of measurement elements (termed as “voxels” hereinafter and discussed in conjunction with FIGS. 4B and 6D) formed by scanning unit 125. Once scanning unit 125 reaches the opposing side of the target area, scanning unit 125 is translated back from the second region toward the starting first region of the target area. The imaging member determines another sequence of representative values of the chromophore property for the same or different set of voxels during this second movement. Depending on the requisite resolution of the final images, this translation may be repeated for a pre-determined period of time or for a pre-selected number of repetitions. After completing the scanning process, the imaging member reorganizes multiple sequences of the representative values, provides a two-dimensional spatial distribution of the chromophore properties, and generates the final images of a spatial distribution thereof over the target area.

FIG. 4B is a schematic diagram of images obtained by the scanning unit of FIG. 3 which is linearly translated across the target area according to the present invention. As manifest in the figure, the entire target area is divided into a series of elements, i.e., the “voxels,” where each elongated voxel 151 extends along a voxel axis 153 throughout a substantial or entire height of the target area. Voxels 151 are sequentially arranged in a voxel direction which is substantially parallel with the curvilinear path of movable member 120. It is appreciated that voxels 151 denoted as a, b, c, and h cover homogeneous regions of the target area (i.e., regions without any abnormalities), while voxels 151 designated as d, e, f, and g include such abnormalities therein.

Each voxel 151 represents a small region of the target area of the medium where the imaging member samples the output signal generated by wave detectors 124 and determines a representative value (termed as “voxel value” hereinafter) of the chromophore property by solving wave equations applied to the wave sources and detectors which define the corresponding voxel. For example, the foregoing equations (1), (2), and/or (6) can be applied to calculate absolute or relative values of concentrations of the hemoglobins and/or oxygen saturation spatially averaged over each of the voxels. That is, the imaging module spatially groups the output signal generated by wave detectors 124 for each voxel 151, and calculates the spatial average values of such chromophore properties of each voxel 151. It is appreciated that the area-averaged voxel value can be substantially similar or identical to the volume-averaged voxel value as long as wave detectors 124 have the sensitivity range covering a substantially identical depth of the medium throughout the entire target area.

Each voxel 151 generally has identical voxel height throughout the entire target area. For example, when scanning unit 125 is moved along a linear path (or rotated about a center of rotation with a pre-selected radius), the voxel height corresponds to an effective height of scanning unit 125 that is measured along the direction orthogonal to the curvilinear path of movable member 120. However, by moving scanning unit 125 along a curved path or two or more different linear paths, voxels 151 may have various voxel heights. It is preferred, however, that voxels 151 have the identical height throughout the entire target area so that data acquisition and processing procedures may be performed by simpler electric circuitry and/or algorithms.

When scanning unit 125 moves along a path which is orthogonal to its longitudinal axis 127, scanning unit 125 can provide a maximum scanning height. In such embodiment, the voxel height is substantially identical to a height of scanning unit 125 and, in addition, voxel axis 153 becomes substantially parallel with axis 127 of scanning unit 125. Furthermore, because voxels 151 are sequentially arranged by scanning unit 125 during its movement, multiple voxels 151 are sequentially arranged side-by-side along the curvilinear path of movable member 110.

Contrary to the voxel height and voxel axis determined by the physical configurations of voxels 151, the voxel width constitutes the characteristic dimension of voxels 151 and, therefore, may be manipulated according to various criteria including, but not limited to, resolution of the final images, mechanical and electrical characteristics of various parts of optical imaging system 100, and the like. It is appreciated that the voxel width may be a direct indicator of the resolution of the final images, because the imaging member is arranged to determine the representative value of the chromophore property per each voxel 151 and to generate the final images based thereupon. For example, in a high-resolution imaging mode, the imaging member calculates each of the foregoing spatially averaged voxel values at every pre-selected distance along the curvilinear path of movable member 110. Such distance may be manipulated to be less than the width of scanning unit 125 by, e.g., increasing the sampling rate of the data acquisition unit, so that each scanning area of scanning unit 125 may include two or more voxels 151. To the contrary, in a low-resolution imaging mode, the imaging member may be arranged to determine each of the above spatial averaged voxel values at a greater distance along the curvilinear path of movable member 120. Accordingly, each scanning area may be only a fraction of a voxel 125 or, conversely, each voxel 151 may have the width enough to encompass therein one or more scanning areas of scanning unit 125.

It is noted that geometric configuration of voxels 151 is determined by a concerted operation of the scanning unit, actuator member, and/or imaging member. Thus, the voxel configuration, in particular, the characteristic dimension of voxels 151 may be manipulated by adjusting operational characteristics of any of the scanning unit, actuator member, and imaging member. For example, by selecting the desired number of the wave sources and detectors of the scanning unit and by depositing them based on a pre-selected geometric arrangement, each or all of the voxels may be arranged to have pre-selected shapes and sizes. The actuator member may be adjusted to vary the speed of movement of the scanning unit and the contour of the curvilinear path, each of which may result in the voxels having various sizes and/or orientations. The imaging member may also be adjusted to receive and sample the output signals at a fixed, variable or adaptive sampling rate. the imaging member may further be manipulated to define multiple scanning units of the wave sources and detectors by grouping such sensors in a variety of configurations. Thus, it is generally a matter of selection of one skilled in the art to manipulate and synchronize the scanning unit, actuator member, and imaging member in order to generate the voxels having optimum shapes and sizes and arranged along the pre-selected path.

FIG. 4C is an example of a two-dimensional spatial distribution of an output signal generated by a wave detector of FIG. 3 which is linearly translated across the target area according to the present invention. In the figure, the ordinate represents magnitude or amplitude of the output signal generated by wave detectors 125 and the abscissa represents a position of scanning unit 125 along the path of the linear translation or a distance of travel thereof. A two-dimensional distribution of an exemplary output signal 150 manifests that the target area may include at least two distinct portions each of which exhibits different optical characteristics. In a first portion 152, e.g., output signal 150 is substantially flat and maintains substantially identical magnitudes. This portion 152 generally corresponds to the regions a, b, c, and h of the rectangular target area of FIG. 4B and represents the starting and end positions of scanning unit 125. To the contrary, in a second portion 154 interposed between the region a, b, and c and the region h, output signal 150 is relatively curved and has smaller magnitudes which vary according to the position along the target area. This may indicate that an abnormality such as a tumor may exist in second portion 154 of the target area. As will be discussed below, identifying such first and second portions 152, 154 of output signal 150 constitutes a basis of calculating a baseline of output signal 150 and of self-calibrating such output signal 150 for the optical imaging system. It is appreciated that second, curved portion 154 of output signal 150 may have the magnitudes greater than those of first, flat portion 152 when output signal 150 therein has a reversed polarity, when the abnormality has different optical characteristics during various developmental stages, and the like.

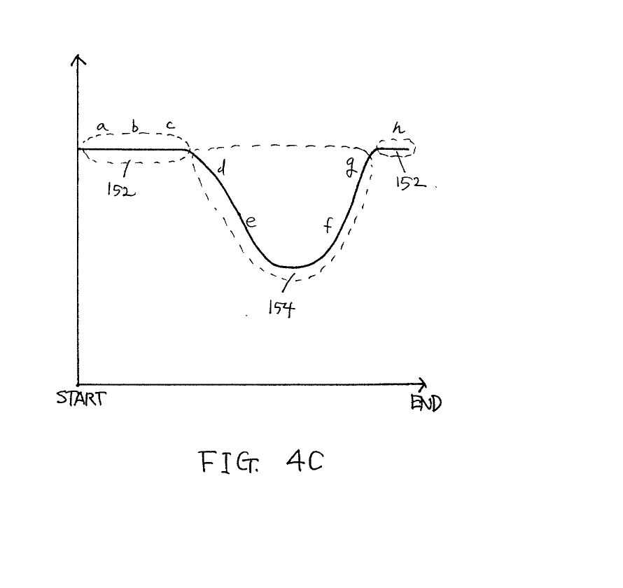

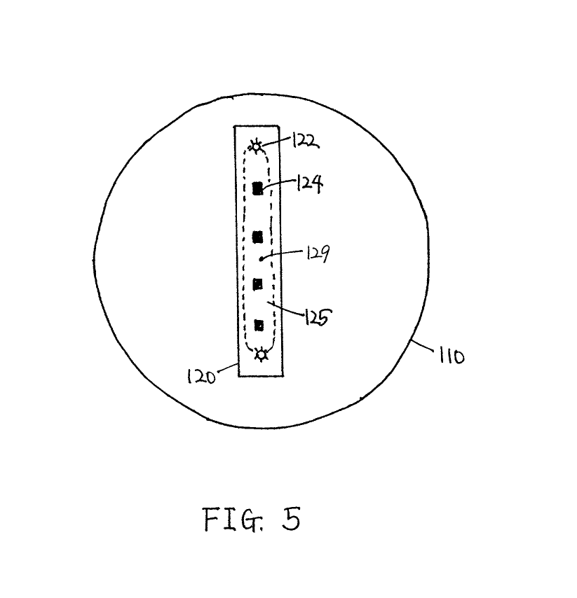

FIG. 5 is another schematic diagram of the scanning unit of FIG. 3 arranged for rotation or revolution according to the present invention. The actuating member is generally arranged to rotate scanning unit 125 about a pre-selected center of location which is, e.g., its mid-point 129. Accordingly, rotations or revolutions of such scanning unit 125 cover an arcuate or circular scanning area having a radius which is substantially identical to one half length of scanning unit 125. Body 110 is generally shaped and sized as an arc or circle so as to accommodate the shape and size of the scanning area defined by scanning unit 125 and to minimize formation of the dead areas thereon.

The actuator member may be arranged to generate different types of rotations or revolutions of the scanning unit. For example, the actuator member may rotate the scanning unit about the center of rotation provided adjacent to one of the edges thereof. Rotations or revolutions of such scanning unit result in an arcuate or a circular scanning area having a diameter which is twice the length of the scanning unit. Alternatively, the actuator member may be arranged to generate two or more movements, rendering the scanning unit define the scanning area comprised of a combination of arcuate and circular areas with different radii and/or different centers of rotation. In addition, the actuator member may also be arranged to manipulate the scanning unit to combine such arcuate or circular movements with linear translations. When it is desired to provide such scanning areas, an optional controller may be provided so as to fine-control the movements of the actuator member along the multiple, pre-selected curvilinear paths.

As described above, the actuator member may generate at least two different movements of the movable member along at least two different curvilinear paths and/or in at least two different curvilinear directions. Such movements may be tailored to satisfy a pre-selected geometric arrangement therebetween. For example, at least a portion of one curvilinear path (or direction) may be substantially transverse to at least a portion of the other curvilinear path (or direction). Such paths may be arranged to be orthogonal to each other as exemplified by the axes of the conventional Cartesian, cylindrical or spherical coordinate systems. In particular, when the target area has a substantially polygonal shape, the actuator member may move the movable member along a first curvilinear path from a first side toward a second opposing side of the target area, to move or reposition it along a second curvilinear path from the second side to the third side thereof, and then to move it along the third curvilinear path from the third side toward the first or other side of such polygonal target area.

In an embodiment of FIGS. 6A to 6D, the actuator member is arranged to generate multiple movements of the scanning unit, e.g., linear translation of the scanning unit (along with the movable member) along the X-axis of the Cartesian coordinate system and clockwise rotation thereof by 90°, followed by another linear translation thereof along the Y-axis. FIGS. 6A, 6B, and 6C are respectively schematic diagrams of the scanning unit of FIG. 3 arranged for such X-translation, 90° rotation, and Y-translation according to the present invention. The optical imaging systems incorporating the embodiment of FIGS. 6A to 6C are substantially identical to those of FIG. 4A, except that the actuator member may move moveable member 120 (e.g., linear translation thereof) independent of the rotation of body 110.

In operation, the actuator member is initialized to position body 110 at its first configuration. Body 110 is placed on the medium to cover at least a substantial portion of the target area, and movable member 120 (along with its scanning unit 125) is positioned in a first region of the target area which is adjacent to one vertical side of the rectangular target area. Wave sources 122 and detectors 124 are carefully positioned to form optical coupling with the first region of the target area so that wave sources 122 can effectively irradiate electromagnetic waves into the first region of the target area and wave detectors 124 can generate the output signal from the first region thereof.

In FIG. 6A, the actuator member (not shown) linearly translates movable member 120 away from the first region of the target area toward an opposing second region along the X-axis (X-translation). Wave sources 122 and detectors 124 are manipulated to maintain the optical couplings with the medium so that wave detectors 124 can generate the output signals representing spatial distribution of the chromophore property during the X-translation. By appropriately manipulating and synchronizing scanning unit 125 with the actuator member, the imaging member (not shown) may sample the output signal at a pre-selected rate. Thus, a set of vertically-extending voxels 161 is defined sequentially along the curvilinear path of scanning unit 125. Because longitudinal axis 127 of scanning unit 125 extends along the Y-axis, voxels 161 also extend along the Y-axis (thus, “Y-extended voxels’), have the height substantially similar to that of scanning unit 125, and have the width which is determined by the speed of the X-translation and the sampling rate of the data acquisition unit of the imaging member. In addition, because the linear translation path of scanning unit 125 is parallel with the X-axis, Y-extended voxels are sequentially arranged along the X-axis. By solving the wave equations based on the spatially averaged output signal in each of the Y-extended voxels, the imaging member calculates the voxel value for each Y-extended voxel.

Once movable member 120 reaches the opposing vertical side of the target area or the vicinity thereof, the actuator member may reposition body 110 to its second configuration by rotating body 110 by 90° in the clockwise direction, as in FIG. 6B, about the center of location disposed at a center of body 110. Such body rotation of 90° results in repositioning movable member 120 (along with scanning unit 125) on or along the upper side of the rectangular target area. The imaging member is synchronized with body 110 and/or the actuator member so as not to sample the output signals during this body rotation.

In FIG. 6C, the actuator member linearly translates movable member 120 (along with scanning unit 125) from the upper side toward an opposing lower side of the target area downwardly along the Y-axis (Y-translation). During the Y-translation, wave sources 122 and detectors 124 are also manipulated to maintain the optical couplings with the medium so that the imaging member can sample the output signal generated by wave detector 124 at a pre-selected rate. Therefore, another set of horizontally-extending voxels 163 is defined sequentially along the curvilinear path of scanning unit 125. Because longitudinal axis 127 of scanning unit 125 is aligned with the X-axis, a set of horizontally-extended voxels 163 are formed along the X-axis (thus, “X-extended voxels’). In addition, because the linear translation path of scanning unit 125 is aligned with the Y-axis, the X-extended voxels are sequentially arranged side by side along the Y-axis. By solving the wave equations based on the spatially averaged output signal in each of the X-extended voxels, the imaging member calculates the voxel value for each X-extended voxel.

Once movable member 120 (and scanning unit 125) reaches the opposing side of the rectangular target area, the scanning process may be terminated. The imaging member then defines a set of cross-voxels 165 by identifying overlapping or intersecting regions between the Y-extended voxels 161 and X-extended voxels 163, and calculates a sequence of cross-voxel values of cross-voxels 165 directly from the voxel values for each pair of Y-extended voxel 161 and X-extended voxel 163 intersecting at each cross-voxel 165. Based on the cross-voxel values, the imaging member produces images of two- or three-dimensional spatial distribution of the properties of the chromophore over at least a substantial portion of the target area.

FIG. 6D is a schematic diagram of images obtained by the scanning unit of FIG. 3 sequentially X-translated, rotated, and Y-translated across the target area according to the present invention. As discussed above, the imaging member defines two orthogonal sets of voxels 161, 163 which intersect each other and define cross-voxels 165. Because each cross-voxel 165 is substantially smaller than Y-extended and X-extended voxels 161, 163, the imaging member can generate high-resolution images of the spatial and/or temporal distribution of the absolute values of the chromophore property.

In general, the characteristic dimensions of voxels 161, 163 such as widths of vertically Y-extended voxels 161 and/or heights of horizontal X-extended voxels 163 may e adjusted by manipulating the speed of the X-translation and Y-translation, respectively, y controlling sampling rate of the output signal, etc. Accordingly, by maintaining the same translational speed during the X- and Y-translations, widths and heights of cross-voxels 165 may become identical, resulting in the square cross-voxels. In the alternative, by employing different speeds during each of the X- and Y-translations and/or by temporally varying such speeds, cross-voxels 161 may have rectangular shapes with different sizes. Thereby, the resolution of the images may be controlled manually or adaptively as well. For example, the speeds of linear translation (or any other movements) may be reduced to obtain smaller rectangular or square cross-voxels from which the imaging member may provide the final images having improved accuracy and enhanced resolution. The characteristic dimensions may similarly be adjusted by manipulating the sampling rate of the output signals by the imaging member.

It is noted that various embodiments may be employed to provide multiple movements of the movable member over the target area. For example, one or more actuator members may be used to provide different movements of the movable member in different directions, e.g., by operating each actuator member to generate a specific movement along a specific curvilinear path and/or by operating a single actuator member which can guide the movable member along different guiding tracks for different curvilinear paths. Although these embodiments may allow meticulous control of the movement of the movable member, they generally require more parts and more elaborate control algorithms. In the alternative, as shown in FIGS. 6A through 6C, the optical imaging system may include a movable body to which both of the actuator member and movable member may be fixedly coupled. By arranging the actuator member to generate a movement of the movable member with respect to the movable body and to generate another movement of the movable body with respect to the target area which is substantially independent of the movement of the movable member, a single actuator member can generate different movements of the movable member along many different curvilinear paths. In addition, the movements of the movable member and movable body may be synchronized to produce a pre-selected movement of the scanning unit over different regions of the target area.

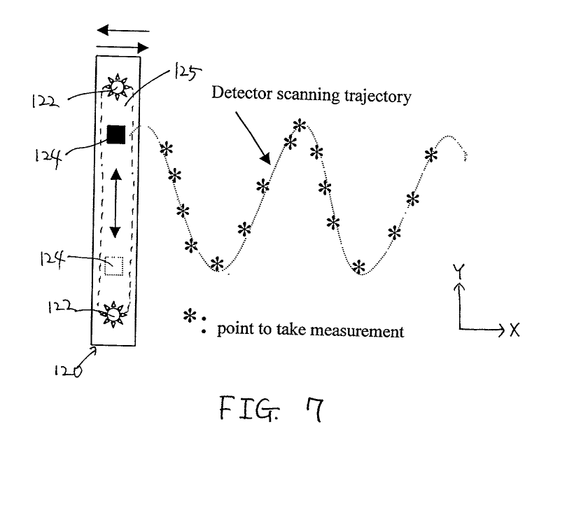

In another aspect of the invention, an optical imaging system includes an actuator member arranged to directly create cross-voxels by generating at least two different movements of a movable member (and/or its scanning unit) simultaneously. This aspect of the invention is described by an exemplary embodiment illustrated in FIG. 7.

FIG. 7 shows a schematic diagram of the scanning unit of FIG. 3 arranged for simultaneous X-Y linear translations according to the present invention. In general, the optical imaging systems incorporating such embodiment are substantially identical to those of FIGS. 4A and 5A, except that the actuator member (not shown) of FIG. 7 is arranged to simultaneously generate a linear translation of movable member 120 along the X-axis and a reciprocation thereof along the Y-axis.

In operation, stationary (or movable) body 110 is placed on a target area of the medium and movable member 120 is positioned in a first region thereof. Wave sources 122 and detectors 124 are also positioned to form appropriate optical coupling with the first region of the target area and turned on to emit electromagnetic waves into and to detect such waves from the target area. The actuator member translates movable member 120 along the X-axis while reciprocating movable member 120 along the Y-axis. Accordingly, movable member 120 (along with scanning unit 125) can scan the target area along a substantially sinusoidal path. It is appreciated that the detailed configuration of such sinusoidal path may be determined by the speed of X-translation as well as that of Y-reciprocation.

Once movable member 120 reaches the opposing side of the target area or the vicinity thereof, an operator may terminate the scanning process of the target area and manually move body 110 to the next target area of the medium for further scanning. In the alternative, the actuator member or an auxiliary motion generating member may be used to mechanically translate and/or rotate body 110 to the next target area as well.

It is noted that accuracy of the output signals may be improved and image resolution may be enhanced by repeating the identical scanning process or performing different scanning processes over the same target area. For example, movable member 120 may be moved back to the starting first region of the target area through the backward X-translation accompanied by the Y-reciprocation thereof. The actuator member may be arranged to move movable member 120 substantially along the same sinusoidal path in the opposite direction and the imaging member may be arranged to sample the output signals at the same measurement locations and sampling rates during the backward movement. By obtaining multiple output signals during the forward and backward movements at each of the voxels, the signal-to-noise ratio of the output signals may be dramatically improved. In the alternative, the actuator member may generate different sinusoidal paths or the imaging member may sample the output signals at different locations and/or at different sampling rates. Accordingly, at least two different sets of voxels may be defined during the forward and backward movements of movable member 110 at each measurement location of the target area. In addition, at least one set of cross-voxels may be generated from multiple sets of voxels extending along different axes, enabling generation of the final images with enhanced resolution. In yet another alternative, more sets of voxels and cross-voxels may also be obtained by arranging body 110 as an article movable with respect to the target area.

Multiple sets of voxels and cross-voxels may be obtained by adjusting or manipulating sampling pattern of the output signals by the imaging member. For example, regardless of the characteristics of curvilinear paths of movable member 120, the imaging member may be synchronized with the actuator member so that the imaging member can sample the output signals at pre-selected locations of the target area. Accordingly, the operator may manipulate the actuator member or imaging member to control the sampling mode of the output signals to adjust the shapes of the voxels and/or cross-voxels, thereby improving resolution of the final images, and so on.

The major advantage attained by the optical imaging system of FIG. 7 is that such systems only needs a minimal number of the wave sources and/or detectors. Contrary to the embodiments shown in FIGS. 4 through 6 where the scanning units preferably have a characteristic dimension (e.g., their height or radius) substantially identical to that of the target area (i.e., the height or radius thereof), the optical imaging system of FIG. 7 defines the scanning unit having the height and/or width substantially less than those of the target area and moves it in at least two directions across the entire portion of the target area, thereby scanning at least a substantial portion thereof. In this respect, the foregoing optical imaging system may even be able to employ a single source-single detector arrangement.

It is appreciated that characteristics of the movement path of the movable member (and/or scanning unit) are not always dispositive of the shapes and/or sizes of the voxels defined thereby. For example, a sinusoidal path of the movable member does not necessarily yield curved voxels arranged along the sinusoidal path of the movable member. When the imaging member samples the output signals at a pre-selected time interval along the sinusoidal path, the voxels may have curved boundaries, varying heights and width, and may be arranged substantially along the sinusoidal path. However, if the imaging member is synchronized with the actuator member to sample the output signals at certain locations, resulting voxels may be manipulated to have substantially identical heights and widths and may be arranged in almost any desirable direction. Furthermore, when the Y-component speed of the movable member (i.e., Y-reciprocation speed) is maintained substantially faster than the X-component thereof (i.e., the X-translation speed), the resulting voxels may have approximately rectangular shapes. By the same token, the voxels may be arranged to be congruent squares, e.g., by synchronizing the imaging member with the actuator member such that the imaging member samples the output signals at every identical horizontal and vertical distance (i.e., identical spatial interval) which may corresponds to different time intervals in the temporal domain.

An actuator member may generate two or more different movements along two or more curvilinear paths so that the imaging member can define the voxels along two or more directions. For example, the embodiment in FIG. 7 allows the imaging member to define the voxels not only along the X-axis but also along the Y-axis. That is, the imaging member defines more than one voxel in the direction which may be orthogonal to the path of the linear translation. By manipulating the speeds along the X- as well as Y-axis and by synchronizing the sampling position or intervals with such movements, the shape and size of the voxels and cross-voxels may also be readily controlled.

It is noted that the voxels obtained by two simultaneous movements of the movable member roughly correspond to the cross-voxels of FIG. 6D obtained by two sequential movements of the movable member. This may be generalized to any movements of the movable member along any curvilinear paths. For example, an actuator member may rotate the movable member while linearly translating (or reciprocating) it along the radial direction. Such an arrangement generally yields a series of spiral layers along the radial direction, where each turn of a spiral layer may contain multiple arcuate voxels. Thus, by maintaining the rotational speed greater than the radial translational speed, the spiral layers approach concentric shells each of which may include multiple arcuate voxels as well.

In yet another aspect of the present invention, an optical imaging system is arranged to directly generate cross-voxels by employing at least one movable wave source and/or detector and at least one stationary wave detector and/or source.

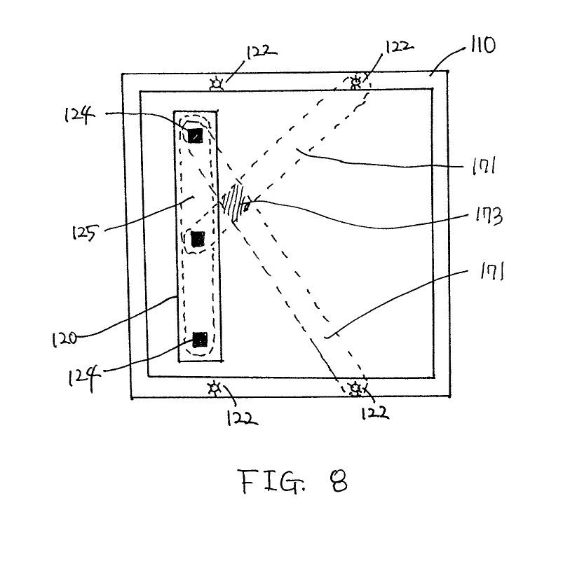

FIG. 8 is a cross-sectional top view of an exemplary scanning unit according to the present invention, in which all four wave sources 122 are disposed along the sides of a stationary body 110, whereas all three wave detectors 124 are implemented to a movable member 120. The actuator member (not shown) generates linear translation or reciprocation of a scanning unit 125 along the X-axis of target area. Therefore, wave sources 122 remain substantially stationary with respect to the target area of the medium, while wave detectors 124 may become movable with respect to wave sources 122 as well as the target area.

In operation, stationary body 110 and movable member 120 are positioned in a first region of the target are so that wave sources 122 form stationary optical coupling with the target area, while wave detectors 124 movably form optical coupling in the first region of the target area. The actuator member translates movable member 120 and its wave detectors 124 from one side to its opposing side of the target area along a linear path which generally corresponds to the X-axis of the target area. Depending upon the data acquisition or sampling rate of the imaging member (not shown), each pair of wave source 122 and detector 124 forms an elongated voxel 171 at an angle with respect to the linear translation path of movable member 120 (or the X-axis). Wave detectors 124 generate representative output signals spatially averaged over an entire area or volume of each elongated voxel 171. The imaging member receives and samples such output signals and determines voxel values for each elongated voxel 171. The imaging member also identifies intersecting portions of two or more voxels and generates cross-voxels 173 thereat. Based on the voxel values of the intersecting voxels, the imaging member calculates cross-voxel values of each of such cross-voxels. Once movable member 120 reaches the opposing side of the target area or the vicinity thereof, the scanning process may be terminated and body 110 is moved to a next target area of the medium for further scanning of thereof. In the alternative, the actuator member may be arranged to repeat the scanning process of the same target area along the identical or different path.

It is appreciated that the geometric relation between stationary wave sources 122 and movable wave detectors 124 varies according to the position in the target area and therefore, scanning unit 125 generally defines extended voxels 171 which have different shapes and sizes during the movement thereof. Such irregular voxels may pose complexity in obtaining a solution of the wave equations applied to scanning unit 125 and, therefore, they are generally less preferred to the ones with substantially identical shapes and sizes. The shape and size differences among extended voxels 171 may be minimized by various arrangements, e.g., by synchronizing the actuator member and imaging member so that the data sampling may be performed at pre-selected positions of the target area, resulting in formation of cross-voxels having predetermined configurations. The shapes and sizes of the cross-voxels and distribution pattern thereof may be controlled by adjusting geometric arrangements between the wave sources and detectors, by varying the speed of their movement, by manipulating the shapes of the curvilinear movement path of the scanning unit, and so on. Accordingly, it is usually a matter of selection of one skilled in the art to find the optimum arrangements for the scanning unit, actuator member, and/or imaging member.

As described above, the accuracy of the output signals and resolution of the images may be enhanced by repeating the same scanning process or performing different scanning processes over the target area. Multiple sets of cross-voxels may be constructed by, e.g., adjusting the sampling pattern of the imaging member or manipulating the path speed of the movement generated by the actuator member. It is noted that, the embodiment of FIG. 8 also requires a minimal number of the wave sources and detectors, and their scanning units may have the heights and widths substantially less than those of the target area.

It is also appreciated that the foregoing optical imaging systems of the present invention can generate the images of two- and/or three-dimensional distribution of the chromophore property on a substantially real time basis. Contrary to every conventional optical imaging system requiring complicated and time-consuming image reconstruction process, the foregoing optical imaging system may generate such images directly from the extended voxel values and/or cross-voxel values of such voxels and/or cross-voxels. For examples, the optical imaging systems of FIGS. 4 through 8 may include real time image construction algorithms regardless of the size of the target area, number of wave sources and detectors, detailed configuration of the curvilinear paths along which the movable and scanning units may travel, and the like. The foregoing optical imaging systems may be readily adjusted for variable resolutions of the images. For example, contrary to the prior art counterparts which require complicated readjustment of the equipment, the foregoing optical imaging system has only to adjust the data sampling rate, speed of movement of the movable member, and so on.

In another aspect of the invention, an optical imaging system may be arranged to include a movable body and a movable member so as to generate images of distribution of chromophore properties in a target area of a physiological medium by moving the movable member within the target area as well as by moving the movable body over different target areas of the medium.

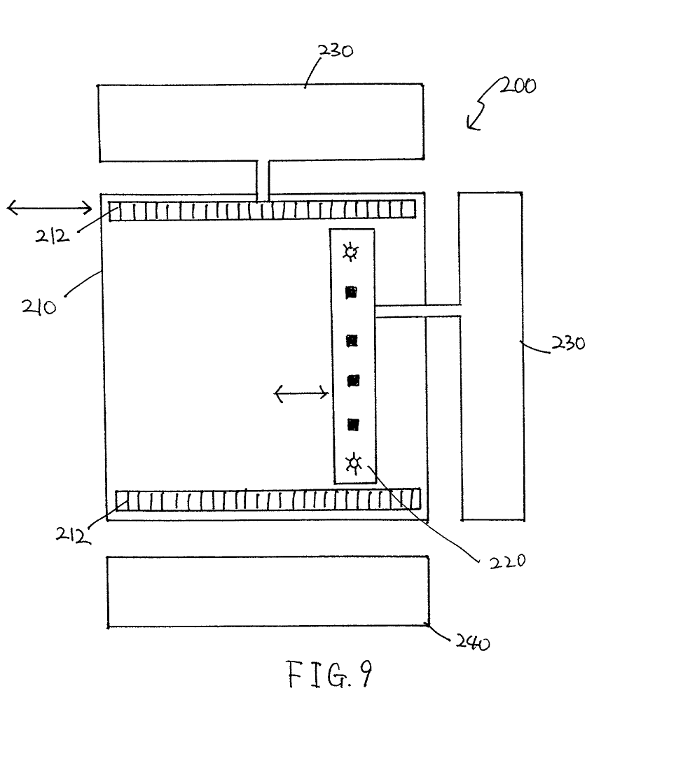

FIG. 9 shows a schematic diagram of another mobile optical imaging system according to the present invention. Such optical imaging system 200 typically includes a movable body 210, movable member 220, and actuator member 230. Movable body 210 is shaped and sized to cover at least a substantial portion of a target area of the medium and preferably encloses at least a portion of movable member 220. Movable body 210 typically includes at least one mobile unit 212 capable of moving movable body 210 over different target areas of the medium. Examples of such mobile units may include, but not limited to, wheels, rollers, caterpillars, and the like. Movable member 220 is arranged to be similar or identical to those described in the foregoing embodiments of FIGS. 1 to 7. For example, movable member 220 may include at least one wave source and at least one wave detector arranged according to any of the foregoing configurations. One or more actuator members 230 operationally may couple with both movable body 210 and movable member 220 to generate at least one primary movement of movable body 210 along at least one primary curvilinear path and at least one secondary movement of movable member 220 along at least one secondary curvilinear path. Actuator member 230 may also generate curvilinear translations, rotations, revolutions or reciprocations of movable body 210 and/or movable member 220 simultaneously or sequentially.

It is appreciated that an optional guiding member may be disposed on the target areas of the medium so that the movable member may travel thereon across multiple target areas. Such guiding member may preferably be made of flexible material or may have a structure so that its shape can conform to different contours of different target areas. For example, a ring-shaped guiding member may be provided to fit around a head or a base portion of a breast of a human subject. The movable member engages the guiding member and moves therealong while allowing the scanning unit to scan around the head or breast. By allowing the movable member to travel along the curvilinear paths of the guiding member with known spatial coordinates at a pre-determined speed, the optical imaging system may readily obtain a continuous two- or three-dimensional distribution of the output signals (or chromophore properties) around the head or breast. In addition, the two-dimensional pattern may readily be combined into the three-dimensional distribution pattern without relying on image markers conventionally required by the prior art optical imaging technology. Therefore, such embodiment also contributes to real-time construction of two- or three-dimensional images of the chromophore properties in the medium.

In a further aspect of the invention, an optical imaging system calculates a baseline or background magnitude (referred to as the “baseline” hereinafter) of an output signal generated by wave detectors. Based on this baseline, the foregoing optical imaging system performs self-calibration of the wave detectors, sensor assembly, optical probe or portable probe of such systems. The self-calibrating optical imaging system may include at least one of the foregoing wave sources, at least one of the foregoing wave detectors, and an imaging member described herein.

The imaging member preferably removes high-frequency noise from the output signal by, e.g., arithmetically, weight- or ensemble-averaging the output signal, and/or processing at least a portion of the output signal through a low-pass filter. The imaging member is arranged to identify different portions or segments of the output signal, each of which exhibits different profile (e.g., flat, linear or curved) and has different (e.g., flat or varying) magnitudes. When the imaging member identifies one or more portions in which the output signal exhibits substantially flat profile and have substantially similar magnitudes, it generally indicates that regions of the target area corresponding to such flat portions of the output signal are predominantly composed of homogeneous material such as normal tissues and cells. The imaging member then calculates the baseline of the output signal by, e.g., arithmetically, geometrically or weight-averaging the magnitudes of the flat or linear portions of the output signal. The imaging member then calculates dimension less or normalized self-calibrated output signal such as, e.g., normalized optical density signals which are defined as ratios of difference signals between the output signal and baseline to the baseline. Such optical density signals may be supplied to the imaging member which then solves a set of wave equations applied to the wave sources and detectors, solutions of which represent the spatially averaged distribution of the properties of the chromophore in different regions of the target area of the medium.

It is preferred that the foregoing self-calibration process be performed on a substantially real-time basis. This implies that, before the movable member of the optical imaging system is moved from the first target area to a next one, the imaging member is preferably arranged to sample the output signals across different regions of the first target area, to calculate the baseline thereof, to generate normalized optical density signals, and to optionally display the output signals, optical density signals, and/or the distribution of the property of the chromophore at each of the voxels defined thereby.

This aspect of the present invention offers several benefits over the prior art. Contrary to prior art optical imaging technology requiring a priori estimation of a medium baseline in a sample medium or in a phantom, the optical imaging system of the present invention estimates a single baseline of the medium and uses that baseline throughout the entire target area and/or medium. Therefore, such optical imaging system obviates any need to estimate multiple baselines without compromising the performance thereof. In addition, the foregoing optical imaging system generates the images of the spatial distribution of the chromophore properties on a substantially real time basis. Furthermore, the probe or its sensors such as the wave sources and detectors of the foregoing optical imaging system do not have to be moved and positioned back and forth between the phantom and the target area. Accordingly, there is no danger of degrading the optical couplings formed between the probe and target area of the medium and, therefore, the resolution of the resulting images can be enhanced.

The flat portion of the output signal or, conversely, the rest of the output signal (i.e., non-flat or curved portion) may be identified by various arrangements. First, one or entire portion of the output signal (or the filtered output signal having an improved signal-to-noise ratio) may be divided into different portions according to a pre-selected threshold value. Such threshold value may be selected as a minimum cut-off value for the flat portion so that all data points of the flat portion may have the magnitudes equal to or greater than the threshold value. In the alternative, the threshold value may be a maximum cut-off value for the non-flat, curved portion so that all data points of the non-flat or curved portion must have magnitudes equal to or less than the threshold magnitude. Regardless of the nature of the threshold value, the output signal may vary in their magnitudes in the flat as well as non-flat portions. Thus, the imaging member may be provided with a secondary cut-off range or a range of deviation, where any data points falling out of the range may not be included in the flat or non-flat portion.

Different methods and/or arrangements may be employed to establish the threshold value. For example, the imaging member may provide an operator with the output signals obtained across different regions of the target area and the operator may manually select the threshold value for the flat portion or non-flat or curved portion of the output signals. The threshold value may also be adaptively determined by identifying a reference value which may be a local (or global) maximum or a local (or global) minimum of the output signal. Once the reference value is identified, the threshold value is readily determined by a pre-selected mathematical equation, e.g., by multiplying (or dividing) the reference value by a pre-selected factor or by subtracting (or adding) a pre-selected offset from (or to) the reference value. In the alternative, the imaging member may calculate a cumulative average of multiple output signals generated by the wave detectors along the curvilinear movement paths of the movable member. The global cumulative average may then be utilized to establish the one or more of the threshold values, reference values, pre-selected factors, and/or pre-selected offsets.

It is appreciated that the imaging member may calculate the baselines for at least two different target areas of the medium. These multiple baselines (referred to as the “local baselines”) may be analyzed to confirm their validity and to select the correct one which is not biased by the presence of abnormal cells or tissues. For example, when the movable member is positioned in a target area free of any abnormalities, the output signal will be flat over the entire region of the target area. The baseline may be easily calculated as the average of the entire output signal. When the target area includes both of the normal and abnormal cells or tissues, the imaging member will divide the output signal into at least two portions, i.e., one flat portion and the other non-flat portion, or will locate such flat portion or segment of the output signal. The baseline may then be calculated as the average of the flat portion of the output signal. However, when the majority portion of or entire target area is composed of the abnormal cells or tissues, the output signal has magnitudes greater than the maximum cut-off value or less than the minimum cut-off value, and may even exhibit a relatively flat profile across the target area. When such target area happens to be the first one to be examined or when the imaging member is arranged to adaptively establish the threshold magnitude based on the average value of the output signal of such target area, an operator may be misled to regard such average value as a correct baseline of the output signal of normal regions. Estimating at least two baselines in at least two different target areas may prevent such misdiagnosis by allowing the operator to manually compare multiple local baselines or by arranging the imaging member to alert the operator upon finding a discrepancy between the baselines obtained from different target areas.

When the local baselines from multiple target areas are not substantially identical or exhibit deviations greater than a pre-selected value, the imaging member may obtain a representative, average or global baseline (“global baseline” hereinafter) and normalize the output signals by such global baseline. Alternatively, an operator or imaging member may select a single baseline from multiple baselines of different target areas and use it as the global baseline. In the alternative, a few selected local baselines or all local baselines may be averaged to calculate the global baseline, where multiple local baselines may be arithmetically, geometrically or weight-averaged to yield the global baseline.

When a global or composite medium image is to be made of multiple local images of multiple target areas, the imaging member may generate each local image based on local baselines of each target areas or based on a single global baseline. For example, the local images of local target areas may be constructed based on each of their local baselines obtained over target areas, and a composite medium image may be obtained by aligning multiple local images obtained by multiple local baselines. In the alternative, the global baseline may be calculated or selected upon which all local images may be based. In general, each approach has its own pros and cons. For example, when a composite image is required around a brain to identify any potential or actual stroke conditions, heterogeneous organs (such as ears, eyes, etc.) and different skull thickness around the brain may yield different local baselines in different target areas around the brain. If the global baseline is calculated from multiple local baselines and used for obtaining all local images, all image pixels will have the identical brightness-scale and/or color-scale across the entire medium. Although such composite medium image may enable a physician to make a comparative diagnosis, he or she may not be able to locate a mild stroke condition which may be hidden in one local target area and overshadowed by the global baseline having magnitude similar to or greater than the mild stroke condition. To the contrary, when the composite image is made of multiple local images each of which is based on individual local baselines, each local image may have its own brightness-scale or color-scale. Although the foregoing mild stroke condition may not be compromised in the local image, the physician may have to analyze each local image separately.

One way of obviating such inconvenience may be to artificially enhance the contrast between normal cells or tissues and abnormalities in each of the local target areas. For example, upon identifying any potential abnormalities, the imaging member may amplify the signals corresponding to such abnormalities so that the amplified signals will not be overshadowed by the magnitude of the global baseline. A special marker or color may be added to such enhanced images to alarm the physician as well. In another example where a composite medium image is required around the breast, some tumors may be as large as or greater than the scanning unit of the optical imaging system or the target area defined thereby. As a result, at least one local image may have the local baseline which may be substantially greater or less than the baseline of normal cells or tissues. To prevent a global baseline from being biased by such abnormal baseline, the imaging member may be arranged to compare individual local baselines obtained from multiple local target areas and not to consider such biased baseline in calculating the global baseline.

Although the above disclosure of the present invention is mainly directed to provide images of a spatial distribution of the chromophore property, the present invention may be applied to generate images of a temporal distribution thereof. As briefly discussed above, the scanning unit of the movable member may be arranged to scan the substantially same region over time. From the differences in the output signals detected at different times over the same region of the medium, the imaging member may calculate temporal changes in the chromophore property of the region and generate images of the temporal distribution pattern of such property. Alternatively, the temporal distribution may be determined and its images may be provided from two or more spatial distributions of chromophore property obtained at different time frames. For example, the movable member and its scanning unit may repeat the scanning process of the target area and calculate the temporal distribution pattern of the chromophore property at each location of the target area. It is appreciated that the temporal changes usually relate to relative changes in the values of the chromophore property. However, once absolute values of the chromophore property may be determined at any reference time frame, preceding or subsequent changes in such property may readily be converted to the absolute values thereof.

It is noted that the foregoing optical imaging systems, optical probes, and methods of the present invention may provide values for the temporal changes in blood or water volume in the target area of the medium. In an exemplary embodiment of obtaining such temporal changes in blood volume in a specific target area of a human subject, the concentration of oxygenated hemoglobin, [HbO], and that of deoxygenated hemoglobin, [Hb], are calculated by a set of equations (1a) and (1b) or by another set of equations (2a) and (2b). Once [Hb] and [HbO] are known, their sum (i.e., total hemoglobin concentration, [HbT], which is the sum of [Hb] and [HbO]) is also calculated. By obtaining the output signals from the wave detectors positioned in the same target area over time, changes in the total hemoglobin concentration is obtained. By assuming that hematocrit (i.e., the volume percentage of the red blood cells in blood) of blood flowing in and out of the target area is maintained at a constant level over time, temporal changes in the blood volume in the target area are directly calculated in terms of temporal changes in [HbT] in the target area. In the alternative, temporal changes in [Hb] and [HbO] may be calculated from the equations (6a) and (6b) and, therefore, temporal changes in [HbT] is obtained as the sum of the changes in [Hb] and [HbO] in the target area.

It is also appreciated that the optical imaging systems, optical probes, and methods of the present invention may be applied to obtain the images of three-dimensional distribution of the chromophore properties in the target area of the medium. As discussed above, electromagnetic waves are irradiated by the wave sources and transmitted through a target volume of the medium which is defined by a target area and a pre-selected depth (or thickness) into the medium. Accordingly, a set of wave equations can be formulated for such three-dimensional target volumes. The output signals generated by the wave detectors are delivered to the imaging member which then solves the wave equations with relevant initial and/or boundary conditions, where such solutions from the wave equations represents the three-dimensional distribution of the chromophore property in the target volume of the medium. To maintain pre-selected resolution of the images, the optical imaging systems or probes thereof preferably include enough number of wave sources and/or detectors arranged to define a larger number of scanning units and voxels in the target volume. Suppose an exemplary optical imaging system includes two wave sources and four wave detectors, and generates the two-dimensional images of a target area with a pre-determined resolution. When a target volume is defined to have an area same as the target area and a pre-selected thickness representing N two-dimensional layers stacked one over the other, such an optical imaging system may probably be required to include approximately 2N wave sources and/or 4N wave detectors to maintain the same resolution as each of two-dimensional layers. The number of requisite wave sources and detectors may be reduced, however, by manipulating the actuator member to generate enough movements of the wave sources and detectors over the target area, preferably in multiple different curvilinear directions. However, the required number of wave sources and detectors is generally inversely proportional to the number or complexity of the movements of the movable member or to the sampling rate of the output signals by the imaging member. Accordingly, the optical imaging system may need fewer number of wave sources and detectors by arranging the actuator member to generate more movements of the scanning unit or by arranging the imaging member to sample the output signals at a higher rate. It is noted, however, that the fundamental resolution of the images obtainable by any optical imaging system is limited by the average “free walk distance” of photons in the physiological medium which is typically about 1 mm. In addition, due to sensitivity limitation and/or electronic and mechanical noise inherent in almost any optical imaging systems, the best-attainable resolution of the optical imaging system may be in the range of a few millimeters or about 1 mm to 5 mm for now. Accordingly, the foregoing voxels which have the dimension less than 1 mm to 5 mm or, more particularly, about 1 mm may not necessarily enhance the resolution of the final images.

The foregoing optical imaging systems, optical probes, and methods of the present invention can be used in both non-invasive and invasive procedures. For example, such optical probes may be non-invasively disposed on the target area on an external surface of the test subject. In the alternative, a miniaturized optical probe may be implemented onto a tip of a catheter and invasively disposed on an internal target area of the subject. The optical imaging systems may be used to determine intensive properties of the chromophores such as concentrations, sums thereof, and ratios thereof, and extensive values thereof such as volume, mass, weight, volumetric flow rate, and mass flow rate thereof.

It is appreciated that the foregoing optical imaging systems, optical probes thereof, and methods therefor may be readily adjusted to provide images of distribution of different chromophores or properties thereof. Because different chromophores generally respond to electromagnetic waves having different wavelengths, the wave sources of such optical imaging systems and probes may be manipulated to irradiate electromagnetic waves interacting with pre-selected chromophores. For example, the near-infrared waves having wavelengths between 600 nm and 1,000 nm, e.g., about 690 nm and 830 nm are suitable to measure the distribution pattern of the hemoglobins and their property. However, the near-infrared waves having wavelengths between 800 nm and 1,000 nm, e.g., about 900 nm, can also be used to measure the distribution pattern of water in the medium. Selection of an optimal wavelength for detecting a particular chromophore generally depends on optical absorption and/or scattering properties of the chromophore, operational characteristics of the wave sources and/or detectors, and the like.

The foregoing optical imaging systems, optical probes, and methods of the present invention may be clinically applied to detect tumors or stroke conditions in human breasts, brains, and any other areas of the human body where the foregoing optical imaging methods such as diffuse optical tomography is applicable. The foregoing optical imaging systems and methods may also be applied to assess blood flow into and out of transplanted organs or extremities and/or autografted or allografted body parts or tissues. The foregoing optical imaging systems and methods may be arranged to substitute, e.g., ultrasonogram, X-rays, EEG, and laser-acoustic diagnostic. Furthermore, such optical imaging systems and methods may be modified to be applicable to various physiological media with complicated photon diffusion and/or with non-flat external surface. It is further noted that the foregoing optical imaging systems, probes thereof, and methods can be applied to conventional optical imaging equipment in which the wave sources and detectors are rather stationarily disposed in their probes.

It is appreciated that the optical imaging systems, optical probes thereof, and methods therefor of the present invention may incorporate or may be applied to other related inventions and embodiments thereof which have been disclosed in the commonly assigned co-pending U.S. application bearing Ser. No. (n/a), entitled “Optical Imaging System with Movable Scanning Unit,” another commonly assigned co-pending U.S. application bearing Ser. No. (n/a), entitled “Self-Calibrating Optical Imaging System,” and yet another commonly assigned co-pending U.S. application bearing Ser. No. (n/a), entitled “Optical Imaging System with Symmetric Optical Probe,” all of which have been filed on Feb. 6, 2001 and all of which are incorporated herein in their entirety by reference.

Following example describes an exemplary optical imaging system, optical probe, and methods thereof according to the present invention. The results indicated that the following exemplary optical imaging system provided reliable and accurate images of two-dimensional distribution of blood volume and oxygen saturation in human breasts.

### EXAMPLE

An exemplary optical imaging system 500 was constructed to obtain images of two-dimensional distribution of blood volume and oxygen saturation in target areas of a female human breast. FIG. 10 is a schematic diagram of a prototype optical imaging system according to the present invention.

Prototype optical imaging system 500 typically included a handle 501 and a main housing 505. Handle 501 was made of poly(vinyl chloride) (PVC) and acrylic stock, and provided with two control switches 503a, 503b for controlling operations of various components of system 500. Main housing 505 included a body 510, movable member 520, actuator member 530, imaging member (not shown), and a pair of guiding tracks 560.

Body 510 was shaped as a substantially square block (3.075″×2.8″×2.63″) and provided with barriers (not shown) along its sides. Body 510 was arranged to movably couple with elongated rectangular movable member 520 (1.5″×2.8″×1.05″) and to linearly translate along a path defined by guiding tracks 560. Movable member 520 included two wave sources 522, S₁ and S₂, each of which was capable of emitting electromagnetic waves having different wavelengths. More particularly, each wave source 522 included two laser diodes, HL8325G and HL6738MG (Thor Labs, Inc., Newton, N.J.), where each laser diode irradiated the electromagnetic waves with wavelengths of 690 nm and 830 nm, respectively. Movable member 520 also included four identical wave detectors 524 such as photo diodes D₁, D₂, D₃, and D₄, (OPT202, Burr-Brown, Tucson, Ariz.) each of which was substantially interposed between wave sources 522. In addition, all wave sources 522 and detectors 524 were spaced substantially linearly at the same distance so that the scanning units defined by wave sources 522 and detectors 524 (e.g., a first scanning unit of S₁, D₁, D₄, and S₂ and a second scanning unit of S₁, D₂, D₃, and S₂) satisfied the foregoing symmetry requirements disclosed in the co-pending '972 application.

A high-resolution linear-actuating-type stepper motor (Model 26000, Hayden Switch and Instrument, Inc., Waterbury, Conn.) and a matching motor controller (Spectrum P.N. 42103, Hayden Switch and Instrument, Inc.) were used as actuator member 530 which was fixedly mounted on body 510. Actuator member 530 was movably engaged with movable member 520 to linearly translate movable member 520 along the paths defined by guiding tracks 560 which were fixedly attached to main housing 505. Precision guides (Model 6725K11, McMaster-Carr Supply, Santa Fe Springs, Calif.) were used as guiding tracks 560. The imaging member was provided inside handle 501 and included a data acquisition card (DICKERED 1200, National Instruments, Austin, Tex.). Main housing 505 was generally made of acrylic stocks and constructed to open at its front face 506. Perspex Non-Glare Acrylic Sheet (Liard Plastics, Santa Clara, Calif.) was installed on front face 506 of main housing 505 and used as a protective screen for protecting wave sources 522 and detectors 524 from mechanical damages.

In operation, movable member 520 was moved to its starting position, i.e., the far-left side of body 510. An operator turned on the main power of system 500 and initialized wave sources 522 and detectors 524 by running scanning system software. A breast of a human subject was prepped and body 505 of optical imaging system 500 was positioned on the breast so that sensors 522, 524 of movable member 520 were positioned in a first region of a first target area of the breast and formed appropriate optical coupling therewith. The operator clicked a first control switch 503a on handle 501. Wave sources 522 irradiated electromagnetic waves having pre-selected wavelengths into the first target area, wave detectors 524 detected such electromagnetic waves from the first target area, and scanning had commenced. Actuator member 530 gradually translated movable member 520 linearly along guiding tracks 560 at a pre-determined speed.

Wave sources 522 were synchronized to ignite each of their laser diodes in a pre-selected sequence. For example, a first laser diode of the wave source, S₁, was arranged to irradiate electromagnetic waves of wavelength 690 nm and wave detectors 524 detected the waves and generated a first set of output signals in response thereto. During this first period of irradiation and detection which generally lasted about 1 msec (with the duty cycle ranging from 1:10 to 1:1,000), all other laser diodes of wave sources S₁ and S₂ were turned off to minimize the interference noises. After completing the irradiation and detection, the first laser diode of the wave source, S₁, was turned off and the first laser diode of the wave source, S₂, was turned on to irradiate electromagnetic waves of the same wavelength, 690 nm. Wave detectors 524 detected the waves and generated a second set of output signals accordingly. Other laser diodes were similarly maintained at their off positions during this second period of irradiation and detection as well. Similar procedures were repeated to the second laser diode of the wave source, S₁, and then to the second laser diode of the wave source S₂, where both second laser diodes sequentially irradiated electromagnetic waves having wavelengths 830 nm.

The imaging member was also synchronized with wave sources 522 and detectors 524 and sampled the foregoing sets of output signals at a pre-selected sampling rate. In particular, the imaging member was arranged to process such output signals by defining a first and second scanning units, where the first scanning unit was comprised of the wave sources, S₁ and S₂, and the wave detectors, D₁ and D₄, while the second scanning unit was made up of the wave sources, S₁ and S₂, and the wave detectors, D₂ and D₃. Both of the first and second scanning units had the source-detector arrangement which satisfied the symmetry requirements of the co-pending '972 application. Therefore, concentrations of oxygenated and deoxygenated hemoglobins were obtained by the equations (1a) to (1d), and the oxygen saturation, SO₂, by the equation (1e). Furthermore, relative blood volume (i.e., temporal changes thereof) was calculated by assessing the changes in concentration of total hemoglobins in different regions of the target area as discussed above.

Actuator member 530 was also synchronized with the foregoing irradiation and detection procedures so that wave sources 522 and detectors 524 scanned an entire first region of the target area (i.e., irradiating electromagnetic waves thereinto, detecting such therefrom, and generating the output signals) before they were moved to the next adjacent region of the target area by actuator member 530. While actuator member 530 translated movable member 520 linearly along the pre-selected path, movable member 520 scanned successive regions of the target area. When movable member 520 reached an opposing end of body 510, actuator member 530 linearly translated movable member 520 backwardly to its starting position, where the irradiation and detection procedures were repeated in the same or different regions of the target area during such backward movement of movable member 520. After the linear reciprocation of movable member 520 ended and scanning procedure was completed, the operator pushed the other control switch 503b to send a signal to the imaging member which then started image construction process and provided two-dimensional images of spatial distribution of the oxygen saturation in the target area and the temporal changes in the blood volume therein.

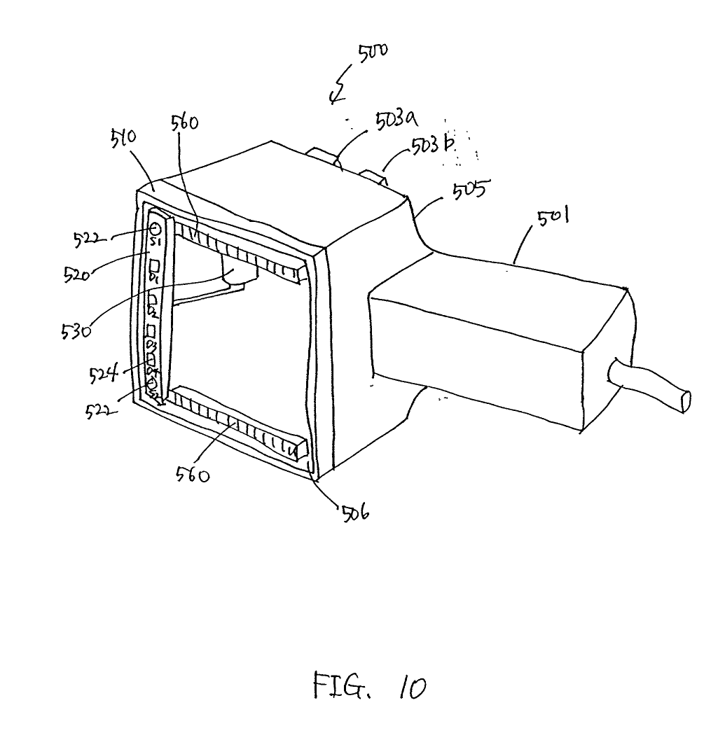

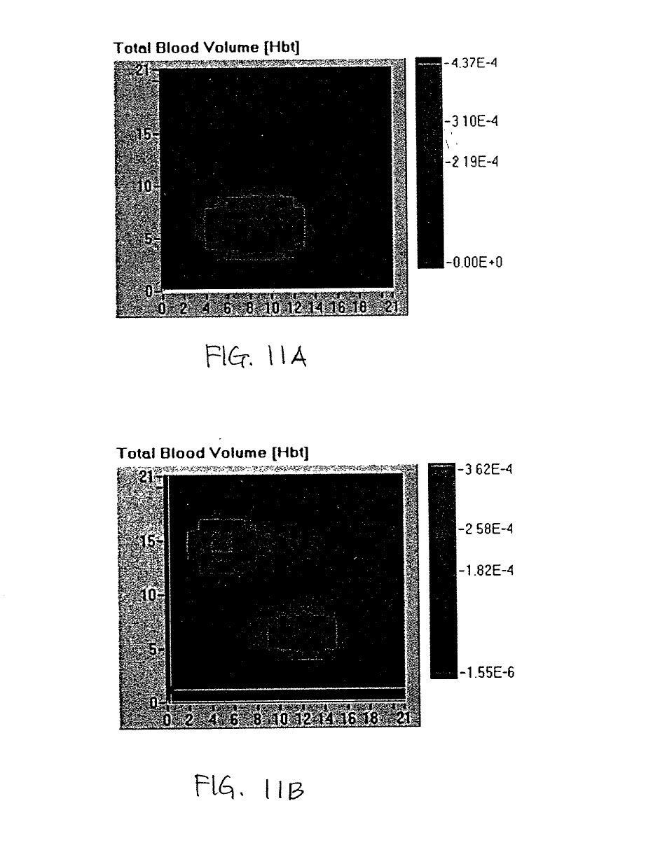

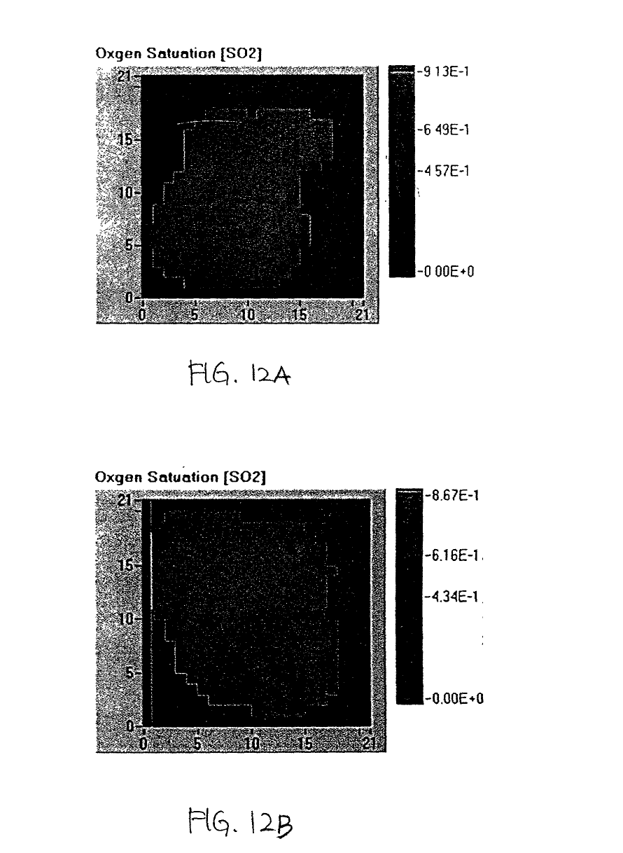

FIGS. 11A and 11B are two-dimensional images of blood volume in normal and abnormal breast tissues, respectively, both measured by the optical imaging system of FIG. 10. In addition, FIGS. 12A and 12B are two-dimensional images of oxygen saturation in normal and abnormal breast tissues, respectively, both measured by the optical imaging system of FIG. 10 according to the present invention. As shown in the figures, the optical imaging system provided that normal tissues had the higher oxygen saturation (e.g., over 70%) in the area with the maximum blood volume. However, the higher oxygen saturation in the corresponding area of the abnormal tissues was as low as 60%.

It is to be understood that, while various embodiments of the invention has been described in conjunction with the detailed description thereof, the foregoing is only intended to illustrate and not to limit the scope of the invention, which is defined by the scope of the appended claims. Other embodiments, aspects, advantages, and modifications are within the scope of the following claims.

What is claimed is:

1. An optical imaging system for generating images of a target area of a physiological medium, said images representing distribution of hemoglobins in the medium, comprising:
at least one movable member with one or more wave sources and one or more wave detectors forming a scanning unit which defines at least one of a scanning area and a scanning volume therearound, the member having and which has a longitudinal axis, said wave source(s) configured to irradiate near-infrared electromagnetic waves into said medium and said wave detector(s) configured to detect said near-infrared electromagnetic waves and to generate output signal in response thereto;
an actuator configured to operationally couple with said movable member and to generate at least one movement of said movable member with respect to said target area along at least one curvilinear path; and
an imaging processor configured to receive said output signal, to define therefrom a plurality of voxels in at least a substantial portion of said target area, to determine said hemoglobin property based on the output signal, and to generate said images of said distribution of hemoglobins, wherein each of said voxels has a characteristic dimension and includes a voxel axis along which it extends.

2. The optical imaging system of claim 1 wherein said characteristic dimension of said voxel is one of a height, length, and width thereof and wherein said characteristic dimension is at least one of substantially parallel with, substantially perpendicular to, and arranged to form a pre-selected angle with said curvilinear path of said movable member.

3. The optical imaging system of claim 1 wherein said voxel axis of said voxel is substantially parallel with said longitudinal axis of said scanning unit.

4. The optical imaging system of claim 1 wherein said voxel has a height which is at least substantially similar to a height of said scanning unit.

5. The optical imaging system of claim 1 wherein said voxel direction of said voxels is substantially parallel with said curvilinear path of said movement of said movable member.

6. The optical imaging system of claim 1 wherein said imaging member is configured to sample said output signal at a pre-selected time interval.

7. The optical imaging system of claim 6 wherein said characteristic dimension of said voxel is at least partially proportional to a speed of said movement of said movable member.

8. The optical imaging system of claim 6 wherein said characteristic dimension of said voxel is at least partially proportional to said sampling time interval of said imaging member.

9. The optical imaging system of claim 1 wherein said imaging member is configured to determine a voxel value for each of said voxels and to generate a sequence of said voxel values arranged in an order of said voxels along said voxel direction, each of said voxel values representing an average of at least one of said output signals and said property of said hemoglobins averaged over each of said voxels.

10. The optical imaging system of claim 9 wherein said average is said property averaged over at least one of said scanning area and scanning volume.

11. The optical imaging system of claim 9 wherein said actuator member is configured to generate at least two movements of said movable member along at least two curvilinear paths and wherein said imaging member is configured to define, during each of said movements, a set of said voxels and a sequence of said voxel values corresponding to said set of said voxels.

12. The optical imaging system of claim 11 wherein said imaging member is configured to define at least one set of a plurality of cross-voxels each of which is defined as an intersecting portion of at least two intersecting voxels each belonging to one of said sets of said voxels.

13. The optical imaging system of claim 12 wherein said imaging member is configured to determine a cross-voxel value for each of said cross-voxels and to generate a sequence of cross-voxel values directly from said voxel values of said intersecting voxels.

14. The optical imaging system of claim 13 wherein each of said cross-voxel values is at least one of an arithmetic sum, arithmetic average, geometric sum, geometric average, weighted sum, and weighted average of said voxel values of said intersecting voxels.

15. The optical imaging system of claim 13 wherein each of said cross-voxel values is at least one of an ensemble sum and ensemble average of said voxel values of said intersecting voxels.

16. The optical imaging system of claim 1 wherein said distribution is at least one of two-dimensional distribution and three-dimensional distribution of said property of said hemoglobins.

17. The optical imaging system of claim 1 wherein said distribution represents at least one of spatial distribution and temporal variation of said property of said hemoglobins.

18. The optical imaging system of claim 1 wherein said property is at least one of spatial changes and temporal changes thereof.

19. The optical imaging system of claim 1 wherein said property is at least one of intensive properties of said hemoglobins including concentration thereof, a sum of said concentrations, a difference of said concentrations, a ratio of said concentrations, and a combination thereof.

20. The optical imaging system of claim 1 wherein said property is at least one of extensive properties of said hemoglobins including volume, mass, volumetric flow rate, and mass flow rate thereof.

21. The optical imaging system of claim 20 wherein said property includes at least one of concentration of oxygenated hemoglobin, concentration of deoxygenated hemoglobin, and oxygen saturation defined as a ratio of said concentration of oxygenated hemoglobin to a sum of said concentrations of deoxygenated hemoglobin and oxygenated hemoglobin.

22. The optical imaging system of claim 1 wherein said electromagnetic waves are at least one of sound waves, near-infrared rays, infrared rays, visible lights, ultraviolet rays, lasers, and photons.

23. An optical imaging system for generating images of a target area of a physiological medium, said images representing distribution of at least one property of one or more chromophores in said medium, said optical imaging system including at least one wave source configured to irradiate electromagnetic waves into said physiological medium and at least one wave detector configured to detect electromagnetic waves and to generate output signal in response thereto, said optical imaging system comprising:
at least one movable member having at least one of said wave source and at least one of said wave detectors, forming a scanning unit, which defines at least one of a scanning area and a scanning volume therearound and which includes a longitudinal axis connecting said wave source and detector;
an actuator configured to operationally couple with said movable member and to generate at least one movement of said movable member with respect to said target area of said medium along at least one curvilinear path; and
an imaging member configured to receive said output signal, to define a set of a plurality of voxels in at least a substantial portion of said target area, to determine said chromophore property, and to generate said images of said distribution of said chromophore property, wherein each of said voxels has a characteristic dimension and includes a voxel axis along which it extends.

24. The optical imaging system of claim 23 wherein said chromophore includes at least one a solvent of said medium, a solute dissolved in said medium, and a substance included in said medium, each of which is configured to interact with said electromagnetic waves irradiated by said wave source and transmitted through said medium.

25. The optical imaging system of claim 23 wherein said chromophore includes at least one of a cytochrome, cytosome, cytosol, enzyme, hormone, neurotransmitter, chemical or chemotransmitter, protein, cholesterol, apoprotein, lipid, carbohydrate, blood cell, water, and hemoglobins including oxygenated and deoxygenated hemoglobin.

26. An optical imaging system configured to generate images of a target area of a physiological medium, said images representing distribution of hemoglobin property in said medium, said optical imaging system comprising:
at least one sensor assembly including at least one wave source and at least one wave detector, said wave source configured to irradiate near-infrared electromagnetic waves into said medium and said wave detector configured to detect said near-infrared electromagnetic waves and to generate output signal in response thereto;
a body configured to support at least a portion of said sensor assembly;
an actuator member operationally coupling with at least one of said sensor assembly and body and configured to generate at least one movement of at least one of said sensor assembly and body with respect to said target area of said medium along at least one curvilinear path; and
an imaging member configured to receive said output signal, to define a set of a plurality of voxels in at least a substantial portion of said target area, to determine said hemoglobin property by solving a plurality of wave equations applied to said wave source and detector, and to generate said images of said distribution of said hemoglobin property.

27. The optical imaging system of claim 26 wherein each of said voxels has a characteristic dimension, wherein each of said voxels includes a voxel axis along which said voxel extends, and wherein said voxels are sequentially arranged along a curvilinear voxel direction.

28. The optical imaging system of claim 26 wherein said imaging member is configured to determine voxel values for said voxels and to generate a sequence of said voxel values arranged in an order of said voxels along said voxel direction, each of said voxel values representing an average of said property of said hemoglobins averaged over each of said voxels.

29. The optical imaging system of claim 26 wherein said imaging member is configured to define at least one set of a plurality of cross-voxels each of which is defined as an intersecting portion of at least two intersecting voxels each belonging to one of said sets of different voxels and each extending along a different voxel axis.

30. An optical imaging system for generating images of a target area of a physiological medium, said images representing distribution of at least one property of at least one chromophore in said medium, said optical imaging system including at least one wave source configured to irradiate electromagnetic waves into said physiological medium and at least one wave detector configured to detect electromagnetic waves and to generate output signal in response thereto, said optical imaging system comprising:
at least one portable probe including at least one movable member and an actuator member, wherein said movable member includes at least one of said wave source and detector and wherein said actuator member is configured to operationally couple with said movable member and to generate at least one movement of said movable member along at least one curvilinear path; and
a console including an imaging member configured to receive said output signal, to define a set of a plurality of voxels in said target area, to determine said property of said chromophore by solving a plurality of wave equations applied to said wave source and detector, and to generate said images of said distribution of said chromophore property.

31. An optical imaging system capable of generating images of target areas of a physiological medium wherein said images represent distribution of at least one property of at least one chromophore in said medium, said optical imaging system comprising:
at least one wave source configured to irradiate electromagnetic waves into said target areas of said physiological medium;
at least one wave detector configured to detect electromagnetic waves and to generate output signal in response thereto;
at least one optical probe including at least one movable member in which at least one of said wave source and detector is disposed;
a console operationally coupling with said optical probe and including an imaging member configured to receive said output signal, to define a set of a plurality of voxels in at least substantial portions of said target areas, to determine said chromophore property by solving a plurality of wave equations applied to said wave source and detector, and to generate said images of said distribution of said chromophore property;
an actuator member configured to operationally couple with said movable member and to generate at least one movement of said movable member along at least one curvilinear path; and
a connector member for providing at least one of electrical communication, optical communication, electric power transmission, mechanical power transmission, and data transmission between at least two of said optical probe, console, and actuator member.

32. An optical imaging system capable of generating images of target areas of a physiological medium, said images representing distribution of at least one property of at least one chromophore in said medium, said optical imaging system comprising:
at least two wave sources configured to irradiate electromagnetic waves into said target areas of said medium;
at least two wave detectors configured to generate output signals responsive to electromagnetic waves detected thereby, wherein at least two of said wave sources and at least two of said wave detectors are disposed substantially linearly along a straight line; and
an imaging member configured to receive said output signal, to define a set of a plurality of voxels in at least substantial portions of said target areas, to determine said chromophore property by solving a set of wave equations applied to said wave sources and detectors, and to generate said images of said distribution of said chromophore property.

33. A method for generating images of a target area of a physiological medium by an optical imaging system, said images representing distribution of hemoglobins in said medium, wherein said optical imaging system includes at least one wave source, at least one wave detector, a movable member, and an actuator member, said wave source configured to irradiate near-infrared electromagnetic waves into said target area of said medium, said wave detector configured to detect said near-infrared electromagnetic waves and to generate output signal in response thereto, said movable member configured to include at least one of said wave source and detector, and said actuator member operationally coupling with said movable member, wherein said wave source and detector are configured to define at least one scanning unit having a longitudinal axis connecting said wave source and detector and defining at least one of a scanning area and scanning volume therearound, and wherein said actuator member is configured to generate at least one movement of at least one of said movable member and said scanning unit along at least one curvilinear path, said method comprising the steps of:
placing said movable member on said target area of said medium;
positioning said scanning unit in a first region of said target area;
scanning said first region by irradiating said near-infrared electromagnetic waves thereinto by said wave source and obtaining said output signal therefrom by said wave detector;
manipulating said actuator member to generate said movement in order to move at least one of said movable member and scanning unit from said first region toward another region of said target area of said medium along a first curvilinear path;
defining at least one first set of a plurality of first voxels from said output signal in at least one of said regions of said target area;
determining a first sequence of first voxel values of said first voxels, each first voxel value being a first average of said property averaged over said first voxel; and
generating said images of said distribution of said hemoglobins from said first sequence of said first voxel values.

34. The method of claim 33 further comprising the steps of:
forming optical couplings between said medium and said wave source and detector; and
maintaining said optical couplings during said movement of at least one of said movable member and scanning unit.

35. The method of claim 33 further comprising the steps of:
arranging all of said wave source and detector substantially linearly along a straight line; and
defining said scanning unit having at least one of said scanning area and scanning volume which is less than said target area and target volume, respectively.

36. The method of claim 33 wherein said generating step comprises the step of:
controlling resolution of said images by varying at least one dimension of said first voxels.

37. The method of claim 36 wherein said varying step comprises at least one of the steps of:
adjusting a distance between said wave source and detector;
adjusting geometric arrangement between said wave source and detector;
adjusting at least one of contour, length, and tortuosity of said curvilinear path of said movement of at least one of said movable member and scanning unit;
adjusting a number of said movements of at least one of said movable member and said scanning unit over said target area;
adjusting a speed of said movement of at least one of said movable member and scanning unit; and
adjusting a sampling rate of said output signal.

38. The method of claim 33 further comprising the steps of:
defining at least one second set of a plurality of second voxels in at least one different region of said target area;
determining a second sequence of second voxel values of said second voxels, each second voxel value representing a second average of said property averaged over said second voxel;
defining a first set of a plurality of first cross-voxels each of which is defined as an intersecting portion of at least two intersecting voxels each belonging to a different set of said voxels;
obtaining a first sequence of first cross-voxel values of said first cross-voxels directly from said voxel values of said intersecting voxels; and
generating said images of said distribution of said hemoglobins from said first sequence of said first cross-voxel values.

39. The method of claim 38 wherein said step of obtaining said first sequence of said first cross-voxel values comprises at least one of the steps of:
arithmetically averaging said voxel values of said intersecting voxels;
geometrically averaging said voxel values of said intersecting voxels;
weight-averaging said voxel values of said intersecting voxels; and
ensemble-averaging said voxel values of said intersecting voxels.

40. The method of claim 38 further comprising the steps of:
defining at least one third set of a plurality of third voxels in at least one yet different region of said target area;
determining a third sequence of third voxel values of said third voxels, each third voxel value being a third average of said property averaged over said third voxel;
defining a second set of a plurality of second cross-voxels each defined as an intersecting portion of at least two intersecting voxels each belonging to a different set of said voxels;
obtaining a second sequence of second cross-voxel values of said second cross-voxel directly from said voxel values of said intersecting voxels; and
generating said images of said distribution of said hemoglobins from said second sequence of said second sequence of said second cross-voxel values.

41. The method of claim 40 further comprising the step of:
generating said images of said distribution of said hemoglobins by arranging a plurality of said sequences of said cross-voxel values, thereby improving the resolution of said images.

42. A method for generating images of a target area of a physiological medium by an optical imaging system, said images representing distribution of at least one property of at least one chromophore in said medium, wherein said optical imaging system includes at least one wave source configured to irradiate electromagnetic waves into said medium and at least one wave detector configured to detect electromagnetic waves and to generate output signal in response thereto, said method comprising the steps of:
positioning said wave source and detector in said target area;
defining a first set of first voxels from said output signals;
determining a first sequence of first voxel values of said first voxels, each first voxel value representing a first average of said property averaged over said first voxel;
defining a second set of second voxels from said output signals;
determining a second sequence of second voxel values of said second voxels, each second voxel value representing a second average of said property averaged over said second voxel;
constructing a first set of first cross-voxels each defined as an intersecting portion of at least two intersecting voxels each of which belongs to one of said first and second sets of said first and second voxels, respectively;
calculating a first sequence of first cross-voxel values of said first cross-voxels directly from said voxel values of said intersecting voxels; and
generating said images of said distribution of said chromophore property from said first sequence of said first cross-voxel values.

43. The method of claim 42 wherein at least one of said defining steps comprises the step of defining said set of said voxels per at least one of:
each pre-selected distance along said target area;
each pre-selected sampling interval of said output signal;
each pair of one of said wave sources and one of said wave detectors; and
each scanning unit comprising at least two of said wave sources and at least two of said wave detectors.

44. The method of claim 42 wherein at least one of said defining steps comprises the step of:
adjusting resolution of said images of said distribution of said property by varying at least one dimension of at least one of said voxels and cross-voxels.

45. The method of claim 42 wherein at least one of said determining steps comprises at least one of the steps of:
averaging said property over an area of said voxel; and
averaging said property over a volume of said voxel.

46. The method of claim 42 wherein said calculating step comprises at least one of the steps of:
arithmetically averaging said voxel values of said intersecting voxels;
geometrically averaging said voxel values of said intersecting voxels;
weight-averaging said voxel values of said intersecting voxels; and
ensemble-averaging said voxel values of said intersecting voxels.

47. A method for generating images of a target area of a physiological medium by an optical imaging system, said images representing distribution of at least one property of at least one chromophore in said medium, wherein said optical imaging system includes at least one wave source, at least one wave detector, a movable member, and an actuator member, said wave source configured to irradiate electromagnetic waves into said medium, said wave detector configured to generate output signal in response to said electromagnetic waves detected thereby, said movable member configured to include at least one of said wave source and detector, and said actuator member operationally coupling with said movable member, wherein said wave source and detector are configured to form a movable scanning unit which includes a longitudinal axis connecting said wave source and detector and which defines at least one of a scanning area and scanning volume therearound, and wherein said actuator member is configured to generate at least one movement of at least one of said movable member and scanning unit along at least one curvilinear path, said method comprising the steps of:
placing said movable member on said target area of said medium;
positioning said scanning unit in a first region of said target area;
manipulating said actuator member to generate a first movement of at least one of said movable member and scanning unit from said first region to a second region of said target area along a first curvilinear path;
defining a first set of first voxels from said output signals in at least a portion of said target area;
determining a first sequence of first voxel values of said first voxels, each first voxel value representing a first average of said property averaged over said first voxel;
defining a second set of second voxels from said output signals in at least a portion of said target area;
determining a second sequence of second voxel values of said second voxels, each second voxel value representing a second average of said property averaged over said second voxel;
constructing a set of cross-voxels each of which is defined as an intersecting portion of at least two intersecting voxels each of which belongs to one of said first and second sets of said first and second voxels, respectively;
calculating a sequence of cross-voxel values of said cross-voxels directly from said voxel values of said intersecting voxels; and
generating said images of said distribution of said property directly from said sequence of said cross-voxel values.
